(19) United States
(12) Patent Application Publication
(10) Pub. No.: US 2024/0270858 A1
(43) Pub. Date: Aug. 15, 2024

(54) THERAPEUTICS AND METHODS FOR TREATING OR AMELIORATING METABOLIC DISORDERS

(71) Applicant: SwiftNovo Therapeutics Inc., Dover, DE (US)

(72) Inventor: Ronghao Li, Foster City, CA (US)

(73) Assignee: SwiftNovo Therapeutics Inc., Dover, DE (US)

(21) Appl. No.: 18/568,113

(22) PCT Filed: Jun. 1, 2022
(86) PCT No.: PCT/IB2022/055142

§ 371 Date: Dec. 7, 2023

Related U.S. Application Data
(60) Provisional application No. 63/208,717, filed on Jun. 9, 2021.

(51) Int. Cl. C07K 16/28; A61K 38/00; A61K 39/00; A61P 1/16; C07K 14/605

(52) U.S. Cl. CPC C07K 16/2869; A61P 1/16; C07K 14/605; A61K 38/00; A61K 2039/505; C07K 2317/565; C07K 2317/92

(57) ABSTRACT

Provided are therapeutics and methods for treating a subject with a metabolic disorder.

### CROSS REFERENCE TO RELATED APPLICATION

This application is the U.S. national phase of International Patent Application No. PCT/IB2022/055142 filed on Jun. 1, 2022, which claims priority to U.S. Provisional Application No. 63/208,717 filed on Jun. 9, 2021. The contents of the applications are incorporated herein by reference in their entirety.

### REFERENCE TO A SEQUENCE LISTING

This application incorporates by reference the Sequence Listing submitted in Computer Readable Form as file Seq Listing 191150.00102_ST25, created on May 16, 2022 and containing 88,426 bytes.

### FIELD OF THE INVENTION

This invention relates to therapeutics and methods for treating a subject with a metabolic disorder, such as fatty liver diseases, using an antigen binding protein that specifically binds to a gastric inhibitory peptide receptor or glucose-dependent insulinotropic polypeptide receptor (GIPR).

### BACKGROUND OF THE INVENTION

Glucose-dependent Insulinotropic Polypeptide (GIP) and Glucagon-Like Peptide-1 (GLP-1) are important incretin hormones due to their ability to increase glucose dependent insulin secretion (Campbell J E and Drucker D J, Cell Metab 2013; 17: 819-837). They are important regulators of energy homeostasis, including glucose and lipid metabolism, appetite, and body weight (Ahren B. Diabetes Obes Metab 2011; 13 Suppl 1: 158-166.). GLP-1 based therapeutics, such as liraglutide, exenatide, dulaglutide, albiglutide and recently approved semaglutide, have been developed for the treatment of type 2 diabetes and obesity. GIP has received less attention due to attenuated response by GIP in diabetic patients (Nauck et al. JCI 1993, 91:301-307). No GIP-based therapy has been approved yet.

GIP and GLP-1 are secreted by the gut in response to food intake (Falko et al. J. Clin. Endocrinol. Metab. 1975, 41:260). GIP is a 42-amino acid peptide secreted by K-cells, the enteroendocrine cells located in the upper tract of the small intestine, duodenum and jejunum (Damholt et al. Cell Tissue Res. 1999, 298:287-93), while GLP-1 is a 31-amino acid peptide secreted by L-cells, the enteroendocrine cells located in the lower tract of the small intestine, jejunum and ileum (Damholt et al. Cell Tissue Res. 1999, 298:287-93). Both GLP-1 and GIP are quickly inactivated by DPP-4 mediated cleavage post secretion (Kieffer T J, McIntosh C H, and Pederson R A. Endocrinology 1995, 136: 3585-3596.). GIP binds to GIPR, a Gs-coupled class B GPCR, while GLP-1 binds to GLP-1R, another closed related Gs-coupled class B GPCR (Couvineau et al. Curr Drug Targets. 2012, 1:103-15.). Upon the binding of GIP and GLP-1 to their receptors, they activate Gas and lead to increase in cAMP level (Tseng et al. Endocrinology. 2000, 141(3):947-52).

GIPR is expressed in the pancreatic islets, adipose tissue, heart, pituitary, adrenal cortex, and certain regions of the brain (Usdin et al. Endocrinology, 1993, 133:2861-2870). In pancreas, GIP stimulates glucose-dependent insulin secretion. More recently, GIP has been shown to stimulate glucagon secretion, which may contribute to the postprandial hyperglycemia in type 2 diabetic patients (Lund et al. Am J Physiol Endocrinol Metab. 2011, 300(6):E1038-46). In adipose tissues, GIP promotes fatty acid uptake and incorporation along with insulin (Kim et al. JBC 2007, 282:8557). In the brain, GIP might have neuroprotective effects (Faivre et al. Eur J Pharmacol. 2012, 674:294-306.). Human GIPR includes 466 amino acids and its gene is located on chromosome 19q13.3 (Gremlich et al., Diabetes. 1995, 44:1202-8).

GIPR knockout mice are resistant to high fat diet-induced weight gain and have improved insulin sensitivity and lipid profiles. (Miyawaki et al. Nature Med. 2002, 8:738-742). In addition, human genetics studies have associated GIPR with body mass index (BMI) (Speliotes et al. Nat Genet 2010, 42: 937; Okada et al. Nat Genet 2012, 44: 302; Wen et al. Nat Genet 2012, 44: 307, Berndt et al., Nat Genet 2013, 45: 501). Besides the incretin effect, GLP-1 also reduces food intake, delays gastric emptying, and decreases glucagon secretion (Drucker, D J, Diabetes Care 2003, 29(10):2929-40). Long-lasting GLP-1 receptor agonists such as exenatide, liraglutide, dulaglutide, albiglutide, and semaglutide have been developed to treat type 2 diabetes (Nauck et al, Exp Clin Endocrinol Diabetes, 1997, 105: 187-95; Dhillon S, Drugs, 2018, 78(2): 275-284). In addition, GLP-1 receptor agonists are also known to promote body weight loss as well as reduction in blood pressure and plasma cholesterols in patients (Vilsboll et al. BMJ, 2012, 344:d7771), and liraglutide has been approved to treat obesity.

### SUMMARY OF INVENTION

This invention provides therapeutics and methods for treating metabolic disorders.

In one aspect, the invention provides a method of treating a subject with a metabolic disorder. The method comprises administering to the subject a therapeutically effective amount of an antigen binding protein that specifically binds to a protein having an amino acid sequence having at least 90% (e.g., 95, 96, 97, 98, or 99%) amino acid sequence identity to an amino acid sequence of a gastric inhibitory peptide receptor. Examples of the metabolic disorder include a fatty liver disease, such as a disorder of non-alcoholic fatty liver disease and a disorder of nonalcoholic steatohepatitis. In some embodiments, the antigen binding protein is an isolated antibody or antigen-binding fragment.

Accordingly, the invention features an antigen binding protein, isolated antibody or an antigen-binding fragment that specifically binds to the protein having an amino acid sequence having at least 90% amino acid sequence identity to an amino acid sequence of a GIPR. The antigen binding protein, antibody, or antigen-binding fragment comprises (i) a light chain or a light chain variable region that comprises LCDR1, LCDR2 and LCDR3 comprising the respective sequences of a LCDR set selected from the group consisting of SEQ ID NOs: 1-3, SEQ ID NOs: 7-9, SEQ ID NOs: 7, 2, and 3, SEQ ID NOs: 10, 2, and 3, SEQ ID NOs: 12-14, SEQ ID NOs: 18-20, SEQ ID NOs: 24-26, SEQ ID NOs: 30-32, and SEQ ID NOs: 36-38, and/or (ii) a heavy chain or a heavy chain variable region that comprises HCDR1, HCDR2, and HCDR3 comprising the respective sequences of a HCDR set selected from the group consisting of SEQ ID NOs: 4-6, SEQ ID NOs: 4, 11, and 6, SEQ ID NOs: 15-17, SEQ ID NOs: 21-23, SEQ ID NOs: 27-29, SEQ ID NOs: 33-35, SEQ ID NOs: 39-41, SEQ ID NOs: 39, 40, and 42, SEQ ID NOs: 39, 40, and 43, and SEQ ID NOs: 39, 40, and 44.

In one embodiment, the light chain variable region comprises a sequence selected from the group consisting of SEQ ID NOs: 45, 47-50, 57, 59, 61, 63, 65, and 67-69, and the heavy chain variable region comprises a sequence selected from the group consisting of SEQ ID NOs: 46, 51-56, 58, 60, 62, 64, 66, and 70-73.

In another embodiment, the light chain variable region comprises a sequence selected from the group consisting of SEQ ID NOs: 45, 47-50; the heavy chain variable region comprises a sequence selected from the group consisting of SEQ ID NOs: 46, 51-56. Examples include the DB009 antibody and its variants disclosed herein. In yet another embodiment, the light chain variable region comprises a sequence selected from the group consisting of SEQ ID NOs: 65, 67-69, and the heavy chain variable region comprises a sequence selected from the group consisting of SEQ ID NOs: 66, 70-73. Examples include the DB004 antibody and its variants disclosed herein.

In a further embodiment, the light chain variable region and the heavy chain variable region comprise the respective sequences of a set selected from the group consisting of SEQ ID Nos: 57-58, SEQ ID Nos: 59-60, SEQ ID NOs: 61-62, and SEQ ID NOs: 63-64. Examples include the DB010, DB011, DB012, and DB013 antibodies and their variants disclosed herein. In some embodiments, the light chain comprises a sequence selected from the group consisting of SEQ ID NOs: 74 and 78 and the heavy chain comprises a sequence selected from the group consisting of SEQ ID NOs: 76 and 80. In one embodiment, the light chain and the heavy chain comprise the respective sequences of a set selected from the group consisting of SEQ ID NOs: 74 and 76, and SEQ ID NOs: 78 and 80.

The above-described antigen-binding protein, antibody, or antigen-binding fragment may further comprise a variant Fc constant region. The antibody can be a monoclonal antibody, a polyclonal antibody, a recombinant antibody, a human antibody, a humanized antibody, a chimeric antibody, a murine antibody, a multispecific antibody, or an antibody fragment thereof. The antigen-binding protein, antibody or fragment can be conjugated to one or more of a therapeutic agent, a polymer, a detectable label, or an enzyme.

In some embodiments, the antibody or fragment described above is conjugated to a GLP-1 sequence. In one example, the GLP-1 sequence comprises the sequence of SEQ ID NOs: 82 or a functional variant thereof. The GLP-1 sequence can be conjugated to the antibody or the antigen-binding fragment covelantly or non-covelantly. The GLP-1 sequence can be fused in frame to the heavy chain or the light chain or both. The GLP-1 sequence can be fused to the heavy chain or the light chain via a linker sequence. Examples of the linker sequence include any suitable sequence, such as SEQ ID Nos: 83 and 84. Preferably, the GLP-1 sequence is fused to the N-terminus of the heavy chain or the light chain. The GLP-1 conjugated heavy chain can comprise the sequence of SEQ ID NO: 85 or 87, The GLP-1 conjugated light chain can comprise the sequence of SEQ ID NO: 86 or 89. In some embodiments, the light chain and the heavy chain comprise the respective sequences of a set selected from the group consisting of SEQ ID NOs: 78 and 87, and SEQ ID NOs: 89 and 80.

Preferably, the antigen-binding protein, antibody or fragment can bind to the extracellular domain of a human GIPR.

In another aspect, the invention provides an isolated nucleic acid or a set of nucleic acids encoding the antigen-binding protein, or one or more of the CDRs, the heavy or light chain variable region, or antigen-binding portion, of any one of above-described antibodies or antigen-binding fragments. The nucleic acid or nucleic acids can be used to express a polypeptide having one or more sets of the HCDRs or LCDRS, a chain of the antibody or antigen-binding fragment, or the antibody or fragment described above. For this purpose, one can operatively link the nucleic acid or nucleic acids to suitable regulatory sequences to generate an expression vector.

Accordingly, within the scope of this invention is a host cell comprising the vector and a method for producing an antigen binding protein, an antibody, or antigen-binding portion thereof. The method includes: obtaining a cultured host cell comprising a vector comprising a nucleic acid or nucleic acids encoding one or more of the above mentioned antigen binding protein, or CDRs, a polypeptide, a heavy chain variable region or a light chain variable region of the antibody or antigen binding portion as described above; culturing the cell in a medium under conditions permitting expression of a polypeptide encoded by the vector and assembling of an antigen binding protein, antibody or fragment thereof, and purifying the antigen binding protein, antibody or fragment from the cultured cell or the medium of the cell.

The invention further provides a pharmaceutical composition comprising (i) the antigen binding protein, the antibody, or the antigen-binding fragment thereof and (ii) a pharmaceutically acceptable carrier.

Also provided are methods of treating a subject with a metabolic disorder. One method comprises administering to a subject in need thereof therapeutically effective amount of the antigen binding protein, antibody, or antigen-binding fragment or a therapeutically effective amount of the composition. Another method comprises the steps of (a) administering to a subject an effective amount of the nucleic acid(s), the expression vector, or the cell described above and (b) expressing the nucleic acid(s) in the subject. Examples of metabolic disorder include a disorder of fatty liver disease, a disorder of non-alcoholic fatty liver disease, a disorder of nonalcoholic steatohepatitis, and a disorder of glucose metabolism. Examples of the glucose metabolism disorder include hyperglycemia, hyperinsulinemia, glucose intolerance, insulin resistance, diabetes mellitus and obesity.

The method described above can further comprise administering to the subject a second therapeutic agent, such as a Glucagon-Like Peptide-1 (GLP-1) agonist or GLP-1 agonist. Examples of the GLP-1 agonist include one or more selected from the group consisting of liraglutide, exenatide, lixisenatide, dulaglutide, albiglutide, semaglutide, and taspoglutide. The subject can be a mammal, including a human and a non-human mammal. Examples of a non-human mammal include a domesticated mammal, such as a dog, cat, horse, cow, goat, pig, or rabbit.

The details of one or more embodiments of the invention are set forth in the description below. Other features, objectives, and advantages of the invention will be apparent from the description and from the claims.

### BRIEF DESCRIPTION OF THE DRAWINGS

FIG. 1 is a plot showing binding curve of positive antibodies to CHO cells expressing human GIPR.

FIGS. 2A and 2B are diagrams showing in vitro activities of selected anti-GIPR antibodies using GeneBLAzer® GIPR-CRE-bla HEK 293T cell-based assay.

FIG. 3 is a diagram showing a chronic study design in AMLN diet induced mouse NASH model.

FIG. 4 is a diagram showing body weights (g) over time (days) of AMLN mice treated with an anti-GIPR antibody DB007 alone, dulaglutide alone, or DB007 and dulaglutide combination.

FIGS. 5A and 5B are two bar graphs showing liver weight (A) and epididymal fat weight (B) of chow diet control mice and AMLN mice treated with vehicle control, an anti-GIPR antibody DB007 alone, dulaglutide alone, or DB007 and dulaglutide combination at terminal of the study (Day 64).

FIG. 6 is a diagram of bar graphs showing baseline and terminal fasting glucose level of chow diet control mice and AMLN mice treated with vehicle control, an anti-GIPR antibody DB007 alone, dulaglutide alone, or DB007 and dulaglutide combination.

FIGS. 7A and 7B are diagrams of bar graphs showing baseline and terminal plasma total cholesterol level (A) and baseline and terminal plasma triglyceride level (B) of chow diet control mice and AMLN mice treated with vehicle control, an anti-GIPR antibody DB007 alone, dulaglutide alone, or DB007 and dulaglutide combination.

FIGS. 8A and 8B are diagram of bar graphs showing baseline and terminal plasma ALT level (A) and AST level (B) of chow diet control mice and AMLN mice treated with vehicle control, an anti-GIPR antibody DB007 alone, dulaglutide alone, or DB007 and dulaglutide combination.

FIGS. 9A and 9B are diagrams of bar graphs showing terminal liver tissue total cholesterol level (A) and liver tissue triglyceride level (B) of chow diet control mice and AMLN mice treated with vehicle control, an anti-GIPR antibody DB007 alone, dulaglutide alone, or DB007 and dulaglutide combination.

FIGS. 10A and 10B are diagrams of bar graphs showing terminal liver tissue HYP level (A) and liver tissue HCY level (B)o f chow diet control mice and AMLN mice treated with vehicle control, an anti-GIPR antibody DB007 alone, dulaglutide alone, or DB007 and dulaglutide combination.

FIGS. 11A, 11B, 11C and 11D are diagrams of bar graphs showing terminal liver tissue mRNA expression level of a-sma (A), ccl2 (B), col1a1 (C) and tgfb (D) of chow diet control mice and AMLN mice treated with vehicle control, an anti-GIPR antibody DB007 alone, dulaglutide alone, or DB007 and dulaglutide combination.

FIGS. 12A and 12B are micrographs showing histology of H&E staining (A) and sirius red staining (B) of two typical liver tissue sections of chow diet control mice and AMLN mice treated with vehicle control, an anti-GIPR antibody DB007 alone, dulaglutide alone, or DB007 and dulaglutide combination histology data of terminal liver H the bar graphs showing terminal liver tissue mRNA expression level of a-sma (A), ccl2 (B) and col1a1 (C) of AMLN mice treated with an anti-GIPR antibody DB007 alone, dulaglutide alone, or DB007 and dulaglutide combination.

### DETAILED DESCRIPTION OF THE INVENTION

This invention relates to treating a metabolic disorder, such as a fatty liver disease, a disorder of glucose metabolism (e.g., Type 2 diabetes, elevated glucose levels, elevated insulin levels, dyslipidemia, metabolic syndrome (Syndrome X or insulin resistance syndrome), glucosuria, metabolic acidosis, Type 1 diabetes, obesity and conditions exacerbated by obesity) by blocking or interfering with the biological activity of GIP. This invention is based, at least in part, on unexpected anti-GIPR activities of certain antigen binding proteins that specifically bind to GIPR. These antigen-binding proteins (such as monoclonal antibodies or antigen-binding fragments thereof) constitute a novel therapeutic strategy for treating the metabolic disorder. In one embodiment, a therapeutically effective amount of an isolated human GIPR binding protein is administered to a subject in need thereof

### GIPR

The GIP receptor (GIPR) is a member of the secretin-glucagon family of G-protein coupled receptors (GPCRs) having an extracellular N-terminus, seven transmembrane domains and an intracellular C-terminus. The N-terminal extracellular domains of this family of receptors are usually glycosylated and form the recognition and binding domain of the receptor. GIPR is highly expressed in a number of tissues, including the pancreas, gut, adipose tissue, heart, pituitary, adrenal cortex, and brain (Usdin et al., Endocrinology. 1993, 133:2861-2870). Human GIPR comprises 466 amino acids and is encoded by a gene located on chromosome 19q13.3 (Gremlich et al., Diabetes. 1995; 44:1202-8; Volz et al., FEBS Lett. 1995, 373:23-29). Studies have suggested that alternative mRNA splicing results in the production of GIP receptor variants of differing lengths in human, rat and mouse.

Examples of GIPR polypeptide include the following sequences.

The 466 amino acid sequence of human GIPR of NCBI Reference Sequence
NP_0001555 (SEQ ID NO: 91):
MTTSPILQLL LRLSLCGLLL QRAETGSKGQ TAGELYQRWE RYRRECQETL AAAEPPSGLA CNGSEDMYVC
WDYAAPNATA RASCPWYLPW HHHVAAGFVL RQCGSDGQWG LWRDHTQCEN PEKNEAFLDQ RLILERLQVM
YTVGYSLSLA TLLLALLILS LFRRLHCTRN YIHINLFTSF MLRAAAILSR DRLLPRPGPY LGDQALALWN
QALAACRTAQ IVTQYCVGAN YTWLLVEGVY LHSLLVLVGG SEEGHFRYYL LLGWGAPALF VIPWVIVRYL
YENTQCWERN EVKAIWWIIR TPILMTILIN FLIFIRILGI LLSKLRTRQM RCRDYRLRLA RSTLTLVPLL
GVHEVVFAPV TEEQARGALR FAKLGFEIFL SSFQGFLVSV LYCFINKEVQ SEIRRGWHHC RLRRSLGEEQ
RQLPERAFRA LPSGSGPGEV PTSRGLSSGT LPGPGNEASR ELESYC
A 430 amino acid isoform of human GIPR (isoform X1) NCBI Reference Sequence
XP_005258790 (SEQ ID NO: 92):
MTTSPILQLL LRLSLCGLLL QRAETGSKGQ TAGELYQRWE RYRRECQETL AAAEPPSVAA GFVLRQCGSD
GQWGLWRDHT QCENPEKNEA FLDQRLILER LQVMYTVGYS LSLATLLLAL LILSLFRRLH CTRNYIHINL
FTSFMLRAAA ILSRDRLLPR PGPYLGDQAL ALWNQALAAC RTAQIVTQYC VGANYTWLLV EGVYLHSLLV
LVGGSEEGHF RYYLLLGWGA PALFVIPWVI VRYLYENTQC WERNEVKAIW WIIRTPILMT ILINFLIFIR
ILGILLSKLR TRQMRCRDYR LRLARSTLTL VPLLGVHEVV FAPVTEEQAR GALRFAKLGF EIFLSSFQGF
LVSVLYCFIN KEVQSEIRRG WHHCRLRRSL GEEQRQLPER AFRALPSGSG PGEVPTSRGL SSGTLPGPGN
EASRELESYC
A 493 amino acid isoform of human GIPR, produced by alternative splicing has
the sequence of UniProtKB Sequence Identifier P48546-2 (SEQ ID NO: 93):
AAAEPPSGLA CNGSEDMYVC WDYAAPNATA RASCPWYLPW HHHVAAGFVL RQCGSDGQWG LWRDHTQCEN
PEKNEAFLDQ RLILERLQVM YTVGYSLSLA TLLLALLILS LFRRLHCTRN YIHINLFTSF MLRAAAILSR
DRLLPRPGPY LGDQALALWN QALAACRTAQ IVTQYCVGAN YTWLLVEGVY LHSLLVLVGG SEEGHFRYYL
LLGWGAPALF VIPWVIVRYL YENTQCWERN EVKAIWWIIR TPILMTILIN FLIFIRILGI LLSKLRTRQM
RCRDYRLRLA RSTLTLVPLL GVHEVVFAPV TEEQARGALR FAKLGFEIFL SSFQGFLVSV LYCFINKEVG
RDPAAAPALW RRRGTAPPLS AIVSQVQSEI RRGWHHCRLR RSLGEEQRQL PERAFRALPS GSGPGEVPTS
RGLSSGTLPG PGNEASRELE SYC
A 460 amino acid sequence of murine GIPR (NCBI Reference Sequence
NP_001074284; uniprotKB/Swiss-Prot Q0P543-1) (SEQ ID NO: 94):
MPLRLLLLLL WLWGLQWAET DSEGQTTTGE LYQRWEHYGQ ECQKMLETTE PPSGLACNGS FDMYACWNYT
AANTTARVSC PWYLPWFRQV SAGFVFRQCG SDGQWGSWRD HTQCENPEKN GAFQDQTLIL ERLQIMYTVG
YSLSLTTLLL ALLILSLFRR LHCTRNYIHM NLFTSEMLRA AAILTRDQLL PPLGPYTGDQ APTPWNQALA
ACRTAQIMTQ YCVGANYTWL LVEGVYLHHL LVIVGRSEKG HERCYLLLGW GAPALFVIPW VIVRYLRENT
QCWERNEVKA IWWIIRTPIL ITILINFLIF IRILGILVSK LRTRQMRCPD YRLRLARSTL TLVPLLGVHE
VVFAPVTEEQ VEGSLRFAKL AFEIFLSSFQ GFLVSVLYCFINKEVQSEIRQ GWRHRRLRLS LQEQRPRPHQ
ELAPRAVPLS SACREAAVGN ALPSGMLHVP GDEVLESYC
A 230 amino acid isoform of murine GIPR, produced by alternative splicing,
NCBI Reference Sequence: AAI20674 (SEQ ID NO: 95):
MPLRLLLLLL WLWGLQWAET DSEGQTTTGE LYQRWEHYGQ ECQKMLETTE PPSGLACNGS FDMYACWNYT
AANTTARVSC PWYLPWFRQV SAGFVFRQCG SDGQWGSWRD HTQCENPEKN GAFQDQTLIL ERLQIMYTVG
YSLSLTTLLL ALLILSLFRR LHCTRNYIHM NLFTSFMLRA AAILTRDQLL PPLGPYTGDQ APTPWNQVLH
RLLPGGTKTF PIYFRTFPHH
The 129 amino acid sequence of human GIPR extracellular domain (ECD)
Sequence (SEQ ID NO: 96):
MTTSPILQLL LRLSLCGLLL QRAETGSKGQ TAGELYQRWE RYRRECQETL AAAEPPSGLA CNGSEDMYVC
WDYAAPNATA RASCPWYLPW HHHVAAGFVL RQCGSDGQWG LWRDHTQCEN PEKNEAF

As used herein, the terms “GIPR polypeptide” and “GIPR protein” are used interchangeably and mean a naturally-occurring wild-type polypeptide expressed in a mammal, such as a human or a mouse, and includes naturally occurring alleles (e.g., naturally occurring allelic forms of human GIPR protein). For purposes of this disclosure, the term “GIPR polypeptide” can be used interchangeably to refer to any full-length GIPR polypeptide, e.g., SEQ ID NO: 91-96.

The term “GIPR polypeptide” also encompasses a GIPR polypeptide in which a naturally occurring GIPR polypeptide sequence (e.g., SEQ ID NOs: 91-96) has been modified. Such modifications include, but are not limited to, one or more amino acid substitutions, including substitutions with non-naturally occurring amino acids non-naturally-occurring amino acid analogs and amino acid mimetics.

In various embodiments, a GIPR polypeptide comprises an amino acid sequence that is at least about 85% identical to a naturally-occurring GIPR polypeptide (e.g., SEQ ID NOs: 91-96). In other embodiments, a GIPR polypeptide comprises an amino acid sequence that is at least about 90%, or about 95, 96, 97, 98, or 99% identical to a naturally-occurring GIPR polypeptide amino acid sequence (e.g., SEQ ID NOs: 91-96). Such GIPR polypeptides preferably, but need not, possess at least one activity of a wild-type GIPR polypeptide, such as the ability to bind GIP. The present invention also encompasses nucleic acid molecules encoding such GIPR polypeptide sequences.

### Antigen Binding Protein

One aspect of this invention relates to GIPR binding proteins. An “antigen binding protein” as used herein means any protein that specifically binds a specified target antigen, such as a GIPR polypeptide (e.g., a human GIPR polypeptide such as provided above). The term antigen binding protein encompasses intact antibodies that comprise at least two full-length heavy chains and two full-length light chains, as well as derivatives, variants, fragments, and mutations thereof.

Examples of the antigen binding proteins include an antibody or a protein derived from an antibody. Examples of antigen binding proteins include, but are not limited to, monoclonal antibodies, bispecific antibodies, minibodies, domain antibodies such as Nanobodies, synthetic antibodies (sometimes referred to herein as “antibody mimetics”), chimeric antibodies, humanized antibodies, human antibodies, antibody fusions, and portions or fragments of each, respectively. In some instances, the antigen binding protein is an immunological fragment of a complete antibody (e.g., a Fab, a Fab′, a F(ab′)2, and Fv fragments). In other instances, the antigen binding protein is a scFv that uses CDRs from an antibody of the present invention.

In general, a GIPR antigen binding protein is said to “specifically bind” its target antigen GIPR when the antigen binding protein exhibits essentially background binding to non-GIPR molecules. An antigen binding protein that specifically binds GIPR may, however, cross-react with GIPR polypeptides from different species. Typically, a GIPR antigen binding protein specifically binds human GIPR when the dissociation constant (KD) is ≤10⁻⁷ M as measured via a surface plasma resonance technique (e.g., BIACORE, GE-HEALTHCARE Uppsala, Sweden) or KINETIC EXCLUSION ASSAY (KINEXA, Sapidyne, Boise, Id.). A GIPR antigen binding protein specifically binds human GIPR with “high affinity” when the KD is ≤5×10⁻⁹ M, and with “very high affinity” when the KD is ≤5×10⁻¹⁰, as measured using methods described.

An “antigen binding region” means a protein, or a portion of a protein, that specifically binds a specified antigen. For example, that portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”) of an immunoglobulin, single-chain immunoglobulin, or camelid antibody. Certain antigen binding regions also include one or more “framework” regions. A “CDR” is an amino acid sequence that contributes to antigen binding specificity and affinity. “Framework” regions can aid in maintaining the proper conformation of the CDRs to promote binding between the antigen binding region and an antigen.

The antigen binding proteins that are provided are antagonists and typically have one, two, three, four, five, six, seven or all eight of the following characteristics:
(a) ability to prevent or reduce binding of GIP to GIPR, where the levels can be measured, for example, by the methods such as radioactive- or fluorescence-labeled ligand binding study, or by the methods described herein (e.g., cAMP assay or other functional assays). The decrease can be at least 10, 25, 50, 100% or more relative to the pre-treatment levels of SEQ ID NO: 91-96 under comparable conditions.
(b) ability to decrease blood glucose;
(c) ability to increase glucose tolerance;
(d) ability to increase insulin sensitivity;
(e) ability to decrease body weight or reduce body weight gain;
(f) ability to decrease fat mass or decrease inflammation in fat tissue;
(g) ability to decrease fasting insulin levels;
(h) ability to decrease circulating cholesterol levels;
(i) ability to decrease circulating triglyceride levels;
(j) ability to decrease liver steatosis or reduce triglyceride level in liver; and
(k) ability to decrease AST, ALT, and/or ALP levels.

In one embodiment, a GIPR antigen binding protein has one or more of the following activities: (a) binds human GIPR such that KD is ≤200 nM, is ≤150 nM, is ≤100 nM, is ≤50 nM, is ≤10 nM, is ≤5 nM, is ≤2 nM, or is ≤1 nM, e.g., as measured via a surface plasma resonance or kinetic exclusion assay technique, and (b) has a half-life in human serum of at least 3 days.

### Antibodies

The invention disclosed herein involves monoclonal antibodies or antigen-binding fragments thereof. The antibodies are able to treat prophylactically and therapeutically a subject having a metabolic disorder.

Listed in the table below are ID numbers (“DB #”) for a number of exemplary antibodies and their variants, as well as their respective clone names, heavy chains (HC), and light chains (LC). For example, antibody DB004 has the HC of DB004_VH and LC of DB004_VK. Similarly, antibody DB021 (clone name DB004.8) has DB004_VH6 and DB004_VK2 as its HC and LC, respectively. By the same token, antibody DB039 (clone name hDB009.14) is a humanized antibody having DB009.hVH7 and DB009.hVK4 as its HC and LC, respectively.

| Antibody # | Variant Name | HC | LC |
|---|---|---|---|
| DB004 | DB004 | DB004_VH | DB004_VK |
| DB009 | DB009 | mDB009_VH | mDB009_VK |
| DB010 | DB010 | mDB010_VH | mDB010_VK |
| DB011 | DB011 | mDB011_VH | mDB011_VK |
| DB012 | DB012 | mDB012_VH | mDB012_VK |
| DB013 | DB013 | mDB013_VH | mDB013_VK |
| DB014 | DB004.1 | DB004_VH1 | DB004_VK1 |
| DB015 | DB004.2 | DB004_VH2 | DB004_VK1 |
| DB016 | DB004.3 | DB004_VH4 | DB004_VK1 |
| DB017 | DB004.4 | DB004_VH6 | DB004_VK1 |
| DB018 | DB004.5 | DB004_VH1 | DB004_VK2 |
| DB019 | DB004.6 | DB004_VH2 | DB004_VK2 |
| DB020 | DB004.7 | DB004_VH4 | DB004_VK2 |
| DB021 | DB004.8 | DB004_VH6 | DB004_VK2 |
| DB022 | DB004.9 | DB004_VH1 | DB004_VK3 |
| DB023 | DB004.10 | DB004_VH2 | DB004_VK3 |
| DB024 | DB004.11 | DB004_VH4 | DB004_VK3 |
| DB025 | DB004.12 | DB004_VH6 | DB004_VK3 |
| DB026 | hDB009.1 | DB009.hVH6 | DB009.hVK1 |
| DB027 | hDB009.2 | DB009.hVH7 | DB009.hVK1 |
| DB028 | hDB009.3 | DB009.hVH8 | DB009.hVK1 |
| DB029 | hDB009.4 | DB009.hVH9 | DB009.hVK1 |
| DB030 | hDB009.5 | DB009.hVH10 | DB009.hVK1 |
| DB031 | hDB009.6 | DB009.hVH11 | DB009.hVK1 |
| DB032 | hDB009.7 | DB009.hVH6 | DB009.hVK3 |
| DB033 | hDB009.8 | DB009.hVH7 | DB009.hVK3 |
| DB034 | hDB009.9 | DB009.hVH8 | DB009.hVK3 |
| DB035 | hDB009.10 | DB009.hVH9 | DB009.hVK3 |
| DB036 | hDB009.11 | DB009.hVH10 | DB009.hVK3 |
| DB037 | hDB009.12 | DB009.hVH11 | DB009.hVK3 |
| DB038 | hDB009.13 | DB009.hVH6 | DB009.hVK4 |
| DB039 | hDB009.14 | DB009.hVH7 | DB009.hVK4 |
| DB040 | hDB009.15 | DB009.hVH8 | DB009.hVK4 |
| DB041 | hDB009.16 | DB009.hVH9 | DB009.hVK4 |
| DB042 | hDB009.17 | DB009.hVH10 | DB009.hVK4 |
| DB043 | hDB009.18 | DB009.hVH11 | DB009.hVK4 |
| DB044 | hDB009.19 | DB009.hVH6 | DB009.hVK8 |
| DB045 | hDB009.20 | DB009.hVH7 | DB009.hVK8 |
| DB046 | hDB009.21 | DB009.hVH8 | DB009.hVK8 |
| DB047 | hDB009.22 | DB009.hVH9 | DB009.hVK8 |
| DB048 | hDB009.23 | DB009.hVH10 | DB009.hVK8 |
| DB049 | hDB009.24 | DB009.hVH11 | DB009.hVK8 |
| DB050 | | GLP1-DB009.hVH7 | DB009.hVK8 |
| DB051 | | DB009.hVH7 | GLP1-DB009.hVK8 |

Listed below are sequences of LC or HC CDRs 1-3 of the above-described exemplary antibodies, heavy chain, and light chain.

| Antibody | | | |
|---|---|---|---|
| Chain | CDR1 | CDR2 | CDR3 |
| mDB009_VK | SASSVSYMH | SISNLAS | LQRSTYPYT |
| | (SEQ ID NO: 1) | (SEQ ID NO: 2) | (SEQ ID NO: 3) |
| mDB009_VH | SGFSFTGYNMN | NIDPYYGVTDYNLKFKG | ASLLLDY |
| | (SEQ ID NO: 4) | (SEQ ID NO: 5) | (SEQ ID NO: 6) |
| DB009_hVK1 | RASSSVSYLA | SISNRAT | QQRSTYPYT |
| | (SEQ ID NO: 7) | (SEQ ID NO: 8) | (SEQ ID NO: 9) |
| DB009_hVK3 | RASSSVSYLA | SISNLAS | LQRSTYPYT |
| | (SEQ ID NO: 7) | (SEQ ID NO: 2) | (SEQ ID NO: 3) |
| DB009_hVK4 | SASSSVSYLH | SISNLAS | LQRSTYPYT |
| | (SEQ ID NO: 10) | (SEQ ID NO: 2) | (SEQ ID NO: 3) |
| DB009_hVK8 | SASSVSYMH | SISNLAS | LQRSTYPYT |
| | (SEQ ID NO: 1) | (SEQ ID NO: 2) | (SEQ ID NO: 3) |
| DB009_hVH6 | SGFSFTGYNMN | NIDPYYGVTDYNLKFKG | ASLLLDY |
| | (SEQ ID NO: 4) | (SEQ ID NO: 5) | (SEQ ID NO: 6) |
| DB009_hVH7 | SGFSFTGYNMN | NIDPYYGVTDYNLKFKG | ASLLLDY |
| | (SEQ ID NO: 4) | (SEQ ID NO: 5) | (SEQ ID NO: 6) |
| DB009_hVH8 | SGFSFTGYNMN | NINPYYGVTDYNLKFKG | ASLLLDY |
| | (SEQ ID NO: 4) | (SEQ ID NO: 11) | (SEQ ID NO: 6) |
| DB009_hVH9 | SGFSFTGYNMN | NIDPYYGVTDYNLKFKG | ASLLLDY |
| | (SEQ ID NO: 4) | (SEQ ID NO: 5) | (SEQ ID NO: 6) |
| DB009_hVH10 | SGFSFTGYNMN | NIDPYYGVTDYNLKFKG | ASLLLDY |
| | (SEQ ID NO: 4) | (SEQ ID NO: 5) | (SEQ ID NO: 6) |
| DB009_hVH11 | SGFSFTGYNMN | NINPYYGVTDYNLKFKG | ASLLLDY |
| | (SEQ ID NO: 4) | (SEQ ID NO: 11) | (SEQ ID NO: 6) |
| mDB010_VK | RSSQSLENSNGNTFLS | RVSNRFS | LQVTHAPPT |
| | (SEQ ID NO: 12) | (SEQ ID NO: 13) | (SEQ ID NO: 14) |
| mDB010_VH | SGFSLTRYDIS | VIWTDGGTNYNSAFKP | VRGAHYSGDYFDY |
| | (SEQ ID NO: 15) | (SEQ ID NO: 16) | (SEQ ID NO: 17) |
| mDB011_VK | RAGQEINGYLS | AASTLDS | LQYASYPLT |
| | (SEQ ID NO: 18) | (SEQ ID NO: 19) | (SEQ ID NO: 20) |
| mDB011_VH | SGFTFSNYGMS | SISSGGATYYPDTVKG | APYYKYDYGMDY |
| | (SEQ ID NO: 21) | (SEQ ID NO: 22) | (SEQ ID NO: 23) |
| mDB012_VK | RSSQSLLHRNGNIYLH | TVSNRFS | SQSIHVPPT |
| | (SEQ ID NO: 24) | (SEQ ID NO: 25) | (SEQ ID NO: 26) |
| mDB012_VH | SGYIFTNYGMN | WINTYTGEPSYTDDFKG | VKTTGYFMDY |
| | (SEQ ID NO: 27) | (SEQ ID NO: 28) | (SEQ ID NO: 29) |
| mDB013_VK | RASQDISNYLN | STSRLHS | QQRYTLPRT |
| | (SEQ ID NO: 30) | (SEQ ID NO: 31) | (SEQ ID NO: 32) |
| mDB013_VH | SGFTFTSYTLH | YITPYNGETHYNEKFTG | AREAFWYGDS FAMDY |
| | (SEQ ID NO: 33) | (SEQ ID NO: 34) | (SEQ ID NO: 35) |
| DB004_VK | RASQGISGELN | ATSFLES | QQSYTTPLT |
| | (SEQ ID NO: 36) | (SEQ ID NO: 37) | (SEQ ID NO: 38) |
| DB004_VH | GGSFSGYAIS | GVIPIFGIANYAQKFQG | ARTMIVADYYYGMDV |
| | (SEQ ID NO: 39) | (SEQ ID NO: 40) | (SEQ ID NO: 41) |
| DB004_VK1 | RASQGISGFLN | ATSFLES | QQSYTTPLT |
| | (SEQ ID NO: 36) | (SEQ ID NO: 37) | (SEQ ID NO: 38) |
| DB004_VK2 | RASQGISGELN | ATSFLES | QQSYTTPLT |
| | (SEQ ID NO: 36) | (SEQ ID NO: 37) | (SEQ ID NO: 38) |
| DB004_VK3 | RASQGISGELN | ATSFLES | QQSYTTPLT |
| | (SEQ ID NO: 36) | (SEQ ID NO: 37) | (SEQ ID NO: 38) |
| DB004_VH1 | GGSFSGYAIS | GVIPIFGIANYAQKFQG | ARTMIVADYYYGMDV |
| | (SEQ ID NO: 39) | (SEQ ID NO: 40) | (SEQ ID NO: 41) |
| DB004_VH2 | GGSFSGYAIS | GVIPIFGIANYAQKFQG | ARTLIVADYYYGMDV |
| | (SEQ ID NO: 39) | (SEQ ID NO: 40) | (SEQ ID NO: 42) |
| DB004_VH4 | GGSFSGYAIS | GVIPIFGIANYAQKFQG | ARTMIVADYYYGLDV |
| | (SEQ ID NO: 39) | (SEQ ID NO: 40) | (SEQ ID NO: 43) |
| DB004_VH6 | GGSFSGYAIS | GVIPIFGIANYAQKFQG | ARTLIVADYYYGLDV |
| | (SEQ ID NO: 39) | (SEQ ID NO: 40) | (SEQ ID NO: 44) |

Listed below are amino acid sequences of light chain (LC) variable regions and heavy chain (HC) variable regions of several exemplary antibodies.

| Antibody Chain | V-region sequence | Seq ID NO |
|---|---|---|
| mDB009_VK | QIVLTQSPAIMSASPGEKVTITCSASSSVSYMHWFQQKPGTSPKLWIY | 45 |
| | SISNLASGVPARFSGSGSGTSYSLTISRMEAEDAATYYCLQRSTYPYT | |
| | FGGGTKLEIKR | |
| mDB009_VH | EVQLQQSGPELEKPGASVRISCKASGFSFTGYNMNWVKQSNGKSLEWI | 46 |
| | GNIDPYYGVTDYNLKFKGKATLTVDKSSSTAYMELKSLTSEDSAVYYC | |
| | ASLLLDYWGQGTTLTVSS | |
| DB009_hVK1 | DIVLTQSPATLSLSPGERATLSCRASSSVSYLAWYQQKPGQAPRLLIY | 47 |
| | SISNRATGIPARFSGSGSGTDFTLTISSLEPEDFAVYYCQQRSTYPYT | |
| | FGQGTKLEIKR | |
| DB009_hVK3 | DIVLTQSPATLSLSPGERATLSCRASSSVSYLAWYQQKPGQAPRLLIY | 48 |
| | SISNLASGVPARFSGSGSGTDFTLTISSLEPEDFAVYYCLQRSTYPYT | |
| | FGQGTKLEIKR | |
| DB009_hVK4 | DIVLTQSPATLSLSPGERATLSCSASSSVSYLHWYQQKPGQAPRLLIY | 49 |
| | SISNLASGVPARFSGSGSGTDFTLTISSLEPEDFAVYYCLQRSTYPYT | |
| | FGQGTKLEIKR | |
| DB009_hVK8 | DIVLTQSPATLSLSPGERATLSCSASSSVSYMHWFQQKPGQAPRLWIY | 50 |
| | SISNLASGIPARFSGSGSGTDYTLTISSLEPEDFAVYYCLQRSTYPYT | |
| | FGQGTKLEIKR | |
| DB009_hVH6 | EVQLVQSGAEVKKPGSSVKVSCKASGFSFTGYNMNWVRQAPGQGLEWM | 51 |
| | GNIDPYYGVTDYNLKFKGRVTITADKSTSTAYMELSSLRSEDTAVYYC | |
| | ASLLLDYWGQGTLVTVSS | |
| DB009_hVH7 | EVQLVQSGAEVKKPGSSVKVSCKASGFSFTGYNMNWVRQAPGQGLEWI | 52 |
| | GNIDPYYGVTDYNLKFKGKATITADKSTSTAYMELSSLRSEDTAVYYC | |
| | ASLLLDYWGQGTLVTVSS | |
| DB009_hVH8 | EVQLVQSGAEVKKPGSSVKVSCKASGFSFTGYNMNWVRQAPGQGLEWI | 53 |
| | GNINPYYGVTDYNLKFKGKATITADKSTSTAYMELSSLRSEDTAVYYC | |
| | ASLLLDYWGQGTLVTVSS | |
| DB009_hVH9 | EVQLVESGGGLVKPGGSLRLSCAASGFSFTGYNMNWVRQAPGKGLEWI | 54 |
| | GNIDPYYGVTDYNLKFKGRFTISRDDSKNTLYLQMNSLKTEDTAVYYC | |
| | ASLLLDYWGQGTLVTVSS | |
| DB009_hVH10 | EVQLVESGGGLVKPGGSLRLSCAASGFSFTGYNMNWVRQAPGKGLEWI | 55 |
| | GNIDPYYGVTDYNLKFKGKATISRDDSKNTLYLQMNSLKTEDTAVYYC | |
| | ASLLLDYWGQGTLVTVSS | |
| DB009_hVH11 | EVQLVESGGGLVKPGGSLRLSCAASGFSFTGYNMNWVRQAPGKGLEWI | 56 |
| | GNINPYYGVTDYNLKFKGKATISRDDSKNTLYLQMNSLKTEDTAVYYC | |
| | ASLLLDYWGQGTLVTVSS | |
| mDB010_VK | DVVMTQTPLFLPVSLGDQASISCRSSQSLENSNGNTFLSWYLQKPGQS | 57 |
| | PHLLIYRVSNRFSGVLDRFSGSGSGTDFTLKISRVEAEDLGVYFCLQV | |
| | THAPPTFGGGTKLEIKR | |
| mDB010_VH | QVQLKESGPGLVAPSQSLSITCTVSGFSLTRYDISWIRQPPGKGLDWL | 58 |
| | GVIWTDGGTNYNSAFKPRLSISKDSSKSQVFLKMNSLQTDDTAIYYCV | |
| | RGAHYSGDYFDYWGQGTTLTVSS | |
| mDB011_VK | DIQMTQSPSSLSASLGERVSLTCRAGQEINGYLSWLQQKPDGTIKRLI | 59 |
| | YAASTLDSGVPKRFSGTRSGSDYSLTISSLESEDFANYYCLQYASYPL | |
| | TFGAGTKLELKR | |
| mDB011_VH | EVKLMESGGGLVKPGGSLKLSCAASGFTFSNYGMSWVRQTPEKRLEWV | 60 |
| | ASISSGGATYYPDTVKGRFTISRDNARNILYLQMSSLRSEDTAMYYCA | |
| | PYYKYDYGMDYWGQGTSVTVSS | |
| mDB012_VK | DVVMTQTPLSLPVSLGDRVSISCRSSQSLLHRNGNIYLHWYLQKPGQS | 61 |
| | PKLLIYTVSNRFSGVPDRFSGSGSGTDFTLKISRVEAEDLGVYFCSQS | |
| | IHVPPTFGGGSKLEIKR | |
| mDB012_VH | QIQLVQSGPELKKPGETIRISCKASGYIFTNYGMNWVKQTPGKGLKWM | 62 |
| | GWINTYTGEPSYTDDFKGREVESLEISVKTAYLQIDNLRKEDMATYFC | |
| | VKTTGYFMDYWGQGTSVTVSS | |
| mDB013_VK | DIQMTQITSSLSASLGDRVTISCRASQDISNYLNWFQQKPDGTVKLLI | 63 |
| | YSTSRLHSGVPSRFSGSGSGTDYSLTISNLEQEDIATYFCQQRYTLPR | |
| | TFGGGTRLEIKR | |
| mDB013_VH | EVQLQQSGPELVKPGTSMKMSCKASGFTFTSYTLHWVKQKPGQGLEWI | 64 |
| | GYITPYNGETHYNEKFTGKAILTSDKSSSTAYMELSSLTSDDSAVYYC | |
| | AREAFWYGDSFAMDYWGQGTSVTVSS | |
| DB004_VK | AIQLTQSPSALSASVGDSVTITCRASQGISGELNWYQQQPGKAPKLLM | 65 |
| | FATSFLESGVPSRFSGSGSATDFSGSGTDFTLTINNLHPEDFATYYCQ | |
| | QSYTTPLTFGQGTRLEIKR | |
| DB004_VH | QMQLVQSGAEVKKPGSSVKVSCKASGGSFSGYAISWVRQAPGQGLEWM | 66 |
| | GGVIPIFGIANYAQKFQGRGTITADESTRTAYMELSSLRSEDTAVYYC | |
| | ARTMIVADYYYGMDVWGQGTTVTVSS | |
| DB004_VK1 | DIQLTQSPSSLSASVGDRVTITCRASQGISGFLNWYQQKPGKAPKLLM | 67 |
| | FATSFLESGVPSRFSGSGSGTDFTLTISSLQP | |
| | EDFATYYCQQSYTTPLTFGQGTRLEIKR | |
| DB004_VK2 | AIQLTQSPSSLSASVGDRVTITCRASQGISGELNWYQQKPGKAPKLLI | 68 |
| | YATSFLESGVPSRFSGSGSATDFSGSGTDFTL | |
| | TISSLQPEDFATYYCQQSYTTPLTFGQGTRLEIKR | |
| DB004_VK3 | AIQLTQSPSSLSASVGDRVTITCRASQGISGELNWYQQKPGKAPKLLI | 69 |
| | YATSFLESGVPSRFSGSGSGTDFTLTISSLQP | |
| | EDFATYYCQQSYTTPLTFGQGTRLEIKR | |
| DB004_VH1 | QMQLVQSGAEVKKPGSSVKVSCKASGGSFSGYAISWVRQAPGQGLEWM | 70 |
| | GGVIPIFGIANYAQKFQGRVTITADESTSTAYMELSSLRSEDTAVYYC | |
| | ARTMIVADYYYGMDVWGQGTTVTVSS | |
| DB004_VH2 | QMQLVQSGAEVKKPGSSVKVSCKASGGSFSGYAISWVRQAPGQGLEWM | 71 |
| | GGVIPIFGIANYAQKFQGRVTITADESTSTAYMELSSLRSEDTAVYYC | |
| | ARTLIVADYYYGMDVWGQGTTVTVSS | |
| DB004_VH4 | QMQLVQSGAEVKKPGSSVKVSCKASGGSFSGYAISWVRQAPGQGLEWM | 72 |
| | GGVIPIFGIANYAQKFQGRVTITADESTSTAYMELSSLRSEDTAVYYC | |
| | ARTMIVADYYYGLDVWGQGTTVTVSS | |
| DB004_VH6 | QMQLVQSGAEVKKPGSSVKVSCKASGGSFSGYAISWVRQAPGQGLEWM | 73 |
| | GGVIPIFGIANYAQKFQGRVTITADESTSTAYMELSSLRSEDTAVYYC | |
| | ARTLIVADYYYGLDVWGQGTTVTVSS | |

Listed below are amino acid sequences of the light chain (LC) and heavy chain (HC) of DB004-VH6/VK2 (DB021). with DB004 human IgG4/kappa VH6/VK2 variants. Also listed are the corresponding nucleic acid sequences.

LC:
(SEQ ID NO: 74)
MSVPTQVLGLLLLWLTDARCAIQLTQSPSSLSASVGDRVTITCRA
SQGISGELNWYQQKPGKAPKLLIYATSFLESGVPSRFSGSGSATD
FSGSGTDFTLTISSLQPEDFATYYCQQSYTTPLTFGQGTRLEIKR
TVAAPSVFIFPPSDEQLKSGTASVVCLLNNFYPREAKVQWKVDNA
LQSGNSQESVTEQDSKDSTYSLSSTLTLSKADYEKHKVYACEVTH
QGLSSPVTKSENRGEC
(SEQ ID NO: 75)
ATGAGCGTGCCCACCCAGGTGCTGGGCCTGCTGCTGCTGTGGCTG
ACCGACGCCAGATGCGCCATCCAGCTGACCCAGAGCCCCAGCAGC
CTGAGCGCCAGCGTGGGCGACAGAGTGACCATCACCTGCAGAGCC
AGCCAGGGCATCAGCGGCTTCCTGAACTGGTACCAGCAGAAGCCC
GGCAAGGCCCCCAAGCTGCTGATCTACGCCACCAGCTTCCTGGAG
AGCGGCGTGCCCAGCAGATTCAGCGGCAGCGGCAGCGCCACCGAC
TTCAGCGGCAGCGGCACCGACTTCACCCTGACCATCAGCAGCCTG
CAGCCCGAGGACTTCGCCACCTACTACTGCCAGCAGAGCTACACC
ACCCCCCTGACCTTCGGCCAGGGCACCAGACTGGAGATCAAGAGA
ACCGTGGCCGCCCCCAGCGTGTTCATCTTCCCCCCCAGCGACGAG
CAGCTGAAATCTGGAACTGCCTCTGTTGTGTGCCTGCTGAATAAC
TTCTATCCCAGAGAGGCCAAAGTACAGTGGAAGGTGGATAACGCC
CTCCAATCGGGTAACTCCCAGGAGAGTGTCACAGAGCAGGACAGC
AAGGACAGCACCTACAGCCTCAGCAGCACCCTGACGCTGAGCAAA
GCAGACTACGAGAAACACAAAGTCTACGCCTGCGAAGTCACCCAT
CAGGGCCTGAGTTCGCCCGTCACAAAGAGCTTCAACAGGGGAGAG
TGTTGA
HC:
(SEQ ID NO: 76)
MEWSWVFLFFLSVTTGVHSQMQLVQSGAEVKKPGSSVKVSCKASG
GSFSGYAISWVRQAPGQGLEWMGGVIPIFGIANYAQKFQGRVTIT
ADESTSTAYMELSSLRSEDTAVYYCARTLIVADYYYGLDVWGQGT
TVTVSSASTKGPSVFPLAPCSRSTSESTAALGCLVKDYFPEPVTV
SWNSGALTSGVHTFPAVLQSSGLYSLSSVVTVPSSSLGTKTYTCN
VDHKPSNTKVDKRVESKYGPPCPPCPAPEAAGGPSVFLFPPKPKD
TLMISRTPEVTCVVVDVSQEDPEVQFNWYVDGVEVHNAKTKPREE
QFNSTYRVVSVLTVLHQDWLNGKEYKCKVSNKGLPSSIEKTISKA
KGQPREPQVYTLPPSQEEMTKNQVSLTCLVKGFYPSDIAVEWESN
GQPENNYKTTPPVLDSDGSFFLYSRLTVDKSRWQEGNVFSCSVMH
EALHNHYTQKSLSLSLGK
(SEQ ID NO: 77)
ATGGAGTGGAGCTGGGTGTTCCTGTTCTTCCTGAGCGTGACCACC
GGCGTGCACAGCCAGATGCAGCTGGTGCAGAGCGGCGCCGAGGTG
AAGAAGCCCGGCAGCAGCGTGAAGGTGAGCTGCAAGGCCAGCGGC
GGCAGCTTCAGCGGCTACGCCATCAGCTGGGTGAGACAGGCCCCC
GGCCAGGGCCTGGAGTGGATGGGCGGCGTGATCCCCATCTTCGGC
ATCGCCAACTACGCCCAGAAGTTCCAGGGCAGAGTGACCATCACC
GCCGACGAGAGCACCAGCACCGCCTACATGGAGCTGAGCAGCCTG
AGAAGCGAGGACACCGCCGTGTACTACTGCGCCAGAACCCTGATC
GTGGCCGACTACTACTACGGCCTGGACGTGTGGGGCCAGGGCACC
ACCGTGACCGTGAGCAGCGCCAGCACCAAGGGCCCCAGCGTGTTC
CCCCTGGCCCCCTGCAGCAGATCCACCTCCGAGAGCACAGCCGCC
CTGGGCTGCCTGGTCAAGGACTACTTCCCCGAACCGGTGACGGTG
TCGTGGAACTCAGGCGCCCTGACCAGCGGCGTGCACACCTTCCCG
GCTGTCCTACAGTCCTCAGGACTCTACTCCCTCAGCAGCGTGGTG
ACCGTGCCCTCCAGCAGCTTGGGCACGAAGACCTACACCTGCAAC
GTAGATCACAAGCCCAGCAACACCAAGGTGGACAAGAGAGTTGAG
TCCAAATATGGTCCCCCATGCCCACCATGCCCAGCACCTGAGGCC
GCAGGGGGACCATCAGTCTTCCTGTTCCCCCCAAAACCCAAGGAC
ACTCTCATGATCTCCCGGACCCCTGAGGTCACGTGCGTGGTGGTG
GACGTGAGCCAGGAAGACCCCGAGGTCCAGTTCAACTGGTACGTG
GATGGCGTGGAGGTGCATAATGCCAAGACAAAGCCGCGGGAGGAG
CAGTTCAACAGCACGTACCGTGTGGTCAGCGTCCTCACCGTCCTG
CACCAGGACTGGCTGAACGGCAAGGAGTACAAGTGCAAGGTCTCC
AACAAAGGCCTCCCGTCCTCCATCGAGAAAACCATCTCCAAAGCC
AAAGGGCAGCCCCGAGAGCCACAGGTGTACACCCTGCCCCCATCC
CAGGAGGAGATGACCAAGAACCAGGTCAGCCTGACCTGCCTGGTC
AAAGGCTTCTACCCCAGCGACATCGCCGTGGAGTGGGAGAGCAAT
GGGCAGCCGGAGAACAACTACAAGACCACGCCTCCCGTGCTGGAC
TCCGACGGCTCCTTCTTCCTCTACAGCAGGCTAACCGTGGACAAG
AGCAGGTGGCAGGAGGGGAATGTCTTCTCATGCTCCGTGATGCAT
GAGGCTCTGCACAACCACTACACACAGAAGAGCCTCTCCCTGTCT
CTGGGTAAATGA

Listed below are amino acid sequences of the light chain (LC) and heavy chain (HC) of DB009-VH7/VK8 (DB045), with DB009 human IgG4/kappa VH7/VK8 variants. Also listed are the corresponding nucleic acid sequences.

LC:
(SEQ ID NO: 78)
MAWALLLLTLLTQGTGSWADIVLTQSPATLSLSPGERATLSCSAS
SSVSYMHWFQQKPGQAPRLWIYSISNLASGIPARFSGSGSGTDYT
LTISSLEPEDFAVYYCLQRSTYPYTFGQGTKLEIKRTVAAPSVFI
FPPSDEQLKSGTASVVCLLNNFYPREAKVQWKVDNALQSGNSQES
VTEQDSKDSTYSLSSTLTLSKADYEKHKVYACEVTHQGLSSPVTK
SENRGEC
(SEQ ID NO: 79)
ATGGCTTGGGCTCTGCTGCTGCTGACCCTGCTGACTCAAGGCACA
GGCTCTTGGGCTGATATTGTGCTGACCCAGTCTCCTGCCACACTG
TCTTTGAGCCCTGGCGAGAGAGCTACCCTGTCCTGCTCTGCCTCC
TCCTCCGTGTCTTACATGCACTGGTTCCAGCAGAAGCCCGGCCAG
GCTCCTAGACTGTGGATCTACTCCATCTCCAACCTGGCCAGCGGC
ATCCCTGCCAGATTTTCTGGCTCTGGAAGCGGCACCGACTATACC
CTGACCATCAGCTCCCTGGAACCTGAGGACTTCGCCGTGTACTAC
TGCCTGCAGCGGTCCACCTATCCTTACACCTTTGGCCAGGGCACC
AAGCTGGAAATCAAGCGGACAGTGGCCGCTCCTTCCGTGTTCATC
TTCCCACCTTCCGACGAGCAGCTGAAGTCTGGCACAGCCTCTGTC
GTGTGCCTGCTGAACAACTTCTACCCTCGGGAAGCCAAGGTGCAG
TGGAAGGTGGACAATGCCCTGCAGTCCGGCAACTCCCAAGAGTCC
GTGACCGAGCAGGACTCCAAGGACTCTACCTACAGCCTGTCCTCC
ACACTGACCCTGTCCAAGGCCGACTACGAGAAGCACAAGGTGTAC
GCCTGCGAAGTGACCCATCAGGGCCTGTCTAGCCCTGTGACCAAG
TCTTTCAACCGGGGCGAGTGT
HC:
(SEQ ID NO: 80)
MAWALLLLTLLTQGTGSWAEVQLVQSGAEVKKPGSSVKVSCKASG
FSFTGYNMNWVRQAPGQGLEWIGNIDPYYGVTDYNLKFKGKATIT
ADKSTSTAYMELSSLRSEDTAVYYCASLLLDYWGQGTLVTVSSAS
TKGPSVFPLAPCSRSTSESTAALGCLVKDYFPEPVTVSWNSGALT
SGVHTFPAVLQSSGLYSLSSVVTVPSSSLGTKTYTCNVDHKPSNT
KVDKRVESKYGPPCPPCPAPEAAGGPSVFLFPPKPKDTLMISRTP
EVTCVVVDVSQEDPEVQFNWYVDGVEVHNAKTKPREEQFNSTYRV
VSVLTVLHQDWLNGKEYKCKVSNKGLPSSIEKTISKAKGQPREPQ
VYTLPPSQEEMTKNQVSLTCLVKGFYPSDIAVEWESNGQPENNYK
TTPPVLDSDGSFFLYSRLTVDKSRWQEGNVFSCSVMHEALHNHYT
QKSLSLSLGK
(SEQ ID NO: 81)
ATGGCTTGGGCTCTGCTGCTGCTGACCCTGCTGACACAAGGCACA
GGCTCTTGGGCTGAAGTGCAGCTGGTTCAGTCTGGCGCCGAAGTG
AAGAAACCTGGCTCCTCCGTGAAGGTGTCCTGCAAGGCCTCTGGC
TTCTCCTTCACCGGCTACAACATGAACTGGGTCCGACAGGCTCCT
GGACAGGGACTCGAGTGGATCGGCAACATCGACCCTTACTACGGC
GTGACCGACTACAACCTGAAGTTCAAGGGCAAAGCCACCATCACC
GCCGACAAGTCTACCTCCACCGCCTACATGGAACTGTCCAGCCTG
AGATCTGAGGACACCGCCGTGTACTACTGCGCTTCCCTGCTGCTG
GATTATTGGGGCCAGGGCACACTGGTCACCGTGTCCTCTGCTTCT
ACCAAGGGACCCAGCGTGTTCCCTCTGGCTCCTTGCTCCAGATCT
ACCTCCGAGTCTACCGCTGCTCTGGGCTGCCTGGTCAAGGACTAC
TTTCCTGAGCCTGTGACCGTGTCTTGGAACTCTGGCGCTCTGACA
TCCGGCGTGCACACCTTTCCAGCTGTGCTGCAATCCTCCGGCCTG
TACTCTCTGTCCTCCGTCGTGACCGTGCCTTCTAGCTCTCTGGGC
ACCAAGACCTACACCTGTAATGTGGACCACAAGCCTTCCAACACC
AAGGTGGACAAGCGCGTGGAATCTAAGTACGGCCCTCCTTGTCCT
CCATGTCCTGCTCCAGAAGCTGCTGGCGGCCCTTCCGTGTTTCTG
TTCCCTCCAAAGCCTAAGGACACCCTGATGATCTCTCGGACCCCT
GAAGTGACCTGCGTGGTGGTCGATGTGTCCCAAGAGGATCCCGAG
GTGCAGTTCAATTGGTACGTGGACGGCGTGGAAGTGCACAACGCC
AAGACCAAGCCTAGAGAGGAACAGTTCAACTCCACCTACAGAGTG
GTGTCCGTGCTGACAGTGCTGCACCAGGATTGGCTGAACGGCAAA
GAGTACAAGTGCAAGGTGTCCAACAAGGGCCTGCCTTCCAGCATC
GAAAAGACCATCTCCAAGGCCAAGGGCCAGCCTAGGGAACCCCAG
GTTTACACCCTGCCTCCAAGCCAAGAGGAAATGACCAAGAACCAG
GTGTCCCTGACCTGCCTCGTGAAGGGCTTCTACCCTTCCGATATC
GCCGTGGAATGGGAGAGCAATGGCCAGCCAGAGAACAACTACAAG
ACAACCCCTCCTGTGCTGGACTCCGACGGCAGCTTCTTCCTGTAT
TCTCGGCTGACCGTGGACAAGTCCAGATGGCAAGAGGGCAACGTG
TTCTCCTGCTCCGTGATGCACGAGGCCCTGCACAATCACTACACC
CAGAAGTCTCTGTCCCTGAGTCTGGGCAAGTGA

GLP-1 can be conjugated to an antibody described herein. Listed below are amino acid sequences of a GLP-1 sequence used, two linkers used, and two examples of GLP-1 conjugated to variable domains of DB045.

| | | Seq |
|---|---|---|
| | | ID |
| Name | Sequence | NO |
| GLP1 | HGEGTFTSDVSSYLE | 82 |
| | EQAAKEFIAWLVKGG | |
| | G | |
| GS linker 1 | GGGGSGGGGSGGGGS | 83 |
| | A | |
| GS linker 2 | GGSGGGGS | 84 |
| GLP1- | HGEGTFTSDVSSYLE | 85 |
| DB009.hVH7 | EQAAKEFIAWLVKGG | |
| | GGGGGSGGGGSGGGG | |
| | SAEVQLVQSGAEVKK | |
| | PGSSVKVSCKASGFS | |
| | FTGYNMNWVRQAPGQ | |
| | GLEWIGNIDPYYGVT | |
| | DYNLKFKGKATITAD | |
| | KSTSTAYMELSSLRS | |
| | EDTAVYYCASLLLDY | |
| | WGQGTLVTVSS | |
| GLP1- | HGEGTFTSDVSSYLE | 86 |
| DB009.hVK8 | EQAAKEFIAWLVKGG | |
| | GGGSGGGGSDIVLTQ | |
| | SPATLSLSPGERATL | |
| | SCSASSSVSYMHWFQ | |
| | QKPGQAPRLWIYSIS | |
| | NLASGIPARFSGSGS | |
| | GTDYTLTISSLEPED | |
| | FAVYYCLQRSTYPYT | |
| | FGQGTKLEIKR | |

Listed below are amino acid sequences of the light chain (LC) and heavy chain (HC) of GLP1-DB009-VH7/VK8 (DB050), with DB009 human IgG4/kappa VH7/VK8 variants, in which GLP1 is conjugated to the HC. Also listed are the corresponding nucleic acid sequences.

LC:
(SEQ ID NO: 78)
MAWALLLLTLLTQGTGSWADIVLTQSPATLSLSPGERATLSCSAS
SSVSYMHWFQQKPGQAPRLWIYSISNLASGIPARFSGSGSGTDYT
LTISSLEPEDFAVYYCLQRSTYPYTFGQGTKLEIKRTVAAPSVFI
FPPSDEQLKSGTASVVCLLNNFYPREAKVQWKVDNALQSGNSQES
VTEQDSKDSTYSLSSTLTLSKADYEKHKVYACEVTHQGLSSPVTK
SENRGEC
(SEQ ID NO: 79)
ATGGCTTGGGCTCTGCTGCTGCTGACCCTGCTGACTCAAGGCACA
GGCTCTTGGGCTGATATTGTGCTGACCCAGTCTCCTGCCACACTG
TCTTTGAGCCCTGGCGAGAGAGCTACCCTGTCCTGCTCTGCCTCC
TCCTCCGTGTCTTACATGCACTGGTTCCAGCAGAAGCCCGGCCAG
GCTCCTAGACTGTGGATCTACTCCATCTCCAACCTGGCCAGCGGC
ATCCCTGCCAGATTTTCTGGCTCTGGAAGCGGCACCGACTATACC
CTGACCATCAGCTCCCTGGAACCTGAGGACTTCGCCGTGTACTAC
TGCCTGCAGCGGTCCACCTATCCTTACACCTTTGGCCAGGGCACC
AAGCTGGAAATCAAGCGGACAGTGGCCGCTCCTTCCGTGTTCATC
TTCCCACCTTCCGACGAGCAGCTGAAGTCTGGCACAGCCTCTGTC
GTGTGCCTGCTGAACAACTTCTACCCTCGGGAAGCCAAGGTGCAG
TGGAAGGTGGACAATGCCCTGCAGTCCGGCAACTCCCAAGAGTCC
GTGACCGAGCAGGACTCCAAGGACTCTACCTACAGCCTGTCCTCC
ACACTGACCCTGTCCAAGGCCGACTACGAGAAGCACAAGGTGTAC
GCCTGCGAAGTGACCCATCAGGGCCTGTCTAGCCCTGTGACCAAG
TCTTTCAACCGGGGCGAGTGT
HC:
(SEQ ID NO: 87)
MAWALLLLTLLTQGTGSWAHGEGTFTSDVSSYLEEQAAKEFIAWL
VKGGGGGGGSGGGGSGGGGSAEVQLVQSGAEVKKPGSSVKVSCKA
SGFSFTGYNMNWVRQAPGQGLEWIGNIDPYYGVTDYNLKFKGKAT
ITADKSTSTAYMELSSLRSEDTAVYYCASLLLDYWGQGTLVTVSS
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEAAGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK
(SEQ ID NO: 88)
ATGGCTTGGGCTCTGCTGCTGCTGACCCTGCTGACACAAGGCACA
GGCTCTTGGGCTCATGGCGAGGGCACCTTTACCTCCGACGTGTCC
TCCTACCTGGAAGAACAGGCCGCCAAAGAGTTTATCGCCTGGCTG
GTCAAAGGTGGCGGCGGAGGCGGAGGAAGCGGTGGCGGAGGTTCA
GGTGGTGGTGGATCTGCTGAAGTGCAGCTGGTTCAGTCTGGCGCC
GAAGTGAAGAAACCTGGCTCCTCCGTGAAGGTGTCCTGCAAGGCC
TCTGGCTTCTCCTTCACCGGCTACAACATGAACTGGGTCCGACAG
GCTCCTGGACAGGGACTCGAGTGGATCGGCAACATCGACCCTTAC
TACGGCGTGACCGACTACAACCTGAAGTTCAAGGGCAAAGCCACC
ATCACCGCCGACAAGTCTACCTCCACCGCCTACATGGAACTGTCC
AGCCTGAGATCTGAGGACACCGCCGTGTACTACTGCGCTTCCCTG
CTGCTGGATTATTGGGGCCAGGGCACACTGGTCACCGTGTCCTCT
GCTTCTACCAAGGGACCCAGCGTGTTCCCTCTGGCTCCTTGCTCC
AGATCTACCTCCGAGTCTACCGCTGCTCTGGGCTGCCTGGTCAAG
GACTACTTTCCTGAGCCTGTGACCGTGTCTTGGAACTCTGGCGCT
CTGACATCCGGCGTGCACACCTTTCCAGCTGTGCTGCAATCCTCC
GGCCTGTACTCTCTGTCCTCCGTCGTGACCGTGCCTTCTAGCTCT
CTGGGCACCAAGACCTACACCTGTAATGTGGACCACAAGCCTTCC
AACACCAAGGTGGACAAGCGCGTGGAATCTAAGTACGGCCCTCCT
TGTCCTCCATGTCCTGCTCCAGAAGCTGCTGGCGGCCCTTCCGTG
TTTCTGTTCCCTCCAAAGCCTAAGGACACCCTGATGATCTCTCGG
ACCCCTGAAGTGACCTGCGTGGTGGTCGATGTGTCCCAAGAGGAT
CCCGAGGTGCAGTTCAATTGGTACGTGGACGGCGTGGAAGTGCAC
AACGCCAAGACCAAGCCTAGAGAGGAACAGTTCAACTCCACCTAC
AGAGTGGTGTCCGTGCTGACAGTGCTGCACCAGGATTGGCTGAAC
GGCAAAGAGTACAAGTGCAAGGTGTCCAACAAGGGCCTGCCTTCC
AGCATCGAAAAGACCATCTCCAAGGCCAAGGGCCAGCCTAGGGAA
CCCCAGGTTTACACCCTGCCTCCAAGCCAAGAGGAAATGACCAAG
AACCAGGTGTCCCTGACCTGCCTCGTGAAGGGCTTCTACCCTTCC
GATATCGCCGTGGAATGGGAGAGCAATGGCCAGCCAGAGAACAAC
TACAAGACAACCCCTCCTGTGCTGGACTCCGACGGCAGCTTCTTC
CTGTATTCTCGGCTGACCGTGGACAAGTCCAGATGGCAAGAGGGC
AACGTGTTCTCCTGCTCCGTGATGCACGAGGCCCTGCACAATCAC
TACACCCAGAAGTCTCTGTCCCTGAGTCTGGGCAAGTGA

Listed below are amino acid sequences of the light chain (LC) and heavy chain (HC) of DB009-VH7/GLP1-VK8 (DB051), with DB009 human IgG4/kappa VH7/VK8 variants. in which GLP1 is conjugated to the LC. Also listed are the corresponding nucleic acid sequences.

LC:
(SEQ ID NO: 89)
MAWALLLLTLLTQGTGSWAHGEGTFTSDVSSYLEEQAAKEFIAWL
VKGGGGGSGGGGSDIVLTQSPATLSLSPGERATLSCSASSSVSYM
HWFQQKPGQAPRLWIYSISNLASGIPARFSGSGSGTDYTLTISSL
EPEDFAVYYCLQRSTYPYTFGQGTKLEIKRTVAAPSVFIFPPSDE
QLKSGTASVVCLLNNFYPREAKVQWKVDNALQSGNSQESVTEQDS
KDSTYSLSSTLTLSKADYEKHKVYACEVTHQGLSSPVTKSENRGE
C
(SEQ ID NO: 90)
ATGGCTTGGGCTCTGCTGCTGCTGACCCTGCTGACTCAAGGCACA
GGCTCTTGGGCTCATGGCGAGGGCACCTTTACCTCCGACGTGTCC
TCCTACCTGGAAGAACAGGCCGCCAAAGAGTTTATCGCCTGGCTG
GTCAAAGGTGGCGGCGGAGGATCTGGCGGAGGCGGATCTGATATT
GTGCTGACCCAGTCTCCTGCCACACTGTCTTTGAGCCCTGGCGAG
AGAGCTACCCTGTCCTGCTCTGCCTCCTCCTCCGTGTCTTACATG
CACTGGTTCCAGCAGAAGCCCGGCCAGGCTCCTAGACTGTGGATC
TACTCCATCTCCAACCTGGCCAGCGGCATCCCTGCCAGATTTTCT
GGCTCTGGAAGCGGCACCGACTATACCCTGACCATCAGCTCCCTG
GAACCTGAGGACTTCGCCGTGTACTACTGCCTGCAGCGGTCCACC
TATCCTTACACCTTTGGCCAGGGCACCAAGCTGGAAATCAAGCGG
ACAGTGGCCGCTCCTTCCGTGTTCATCTTCCCACCTTCCGACGAG
CAGCTGAAGTCTGGCACAGCCTCTGTCGTGTGCCTGCTGAACAAC
TTCTACCCTCGGGAAGCCAAGGTGCAGTGGAAGGTGGACAATGCC
CTGCAGTCCGGCAACTCCCAAGAGTCCGTGACCGAGCAGGACTCC
AAGGACTCTACCTACAGCCTGTCCTCCACACTGACCCTGTCCAAG
GCCGACTACGAGAAGCACAAGGTGTACGCCTGCGAAGTGACCCAT
CAGGGCCTGTCTAGCCCTGTGACCAAGTCTTTCAACCGGGGCGAG
TGT
HC:
(SEQ ID NO: 80)
MAWALLLLTLLTQGTGSWAEVQLVQSGAEVKKPGSSVKVSCKASG
FSFTGYNMNWVRQAPGQGLEWIGNIDPYYGVTDYNLKFKGKATIT
ADKSTSTAYMELSSLRSEDTAVYYCASLLLDYWGQGTLVTVSSAS
TKGPSVFPLAPCSRSTSESTAALGCLVKDYFPEPVTVSWNSGALT
SGVHTFPAVLQSSGLYSLSSVVTVPSSSLGTKTYTCNVDHKPSNT
KVDKRVESKYGPPCPPCPAPEAAGGPSVFLFPPKPKDTLMISRTP
EVTCVVVDVSQEDPEVQFNWYVDGVEVHNAKTKPREEQFNSTYRV
VSVLTVLHQDWLNGKEYKCKVSNKGLPSSIEKTISKAKGQPREPQ
VYTLPPSQEEMTKNQVSLTCLVKGFYPSDIAVEWESNGQPENNYK
TTPPVLDSDGSFFLYSRLTVDKSRWQEGNVFSCSVMHEALHNHYT
QKSLSLSLGK
(SEQ ID NO: 81)
ATGGCTTGGGCTCTGCTGCTGCTGACCCTGCTGACACAAGGCACA
GGCTCTTGGGCTGAAGTGCAGCTGGTTCAGTCTGGCGCCGAAGTG
AAGAAACCTGGCTCCTCCGTGAAGGTGTCCTGCAAGGCCTCTGGC
TTCTCCTTCACCGGCTACAACATGAACTGGGTCCGACAGGCTCCT
GGACAGGGACTCGAGTGGATCGGCAACATCGACCCTTACTACGGC
GTGACCGACTACAACCTGAAGTTCAAGGGCAAAGCCACCATCACC
GCCGACAAGTCTACCTCCACCGCCTACATGGAACTGTCCAGCCTG
AGATCTGAGGACACCGCCGTGTACTACTGCGCTTCCCTGCTGCTG
GATTATTGGGGCCAGGGCACACTGGTCACCGTGTCCTCTGCTTCT
ACCAAGGGACCCAGCGTGTTCCCTCTGGCTCCTTGCTCCAGATCT
ACCTCCGAGTCTACCGCTGCTCTGGGCTGCCTGGTCAAGGACTAC
TTTCCTGAGCCTGTGACCGTGTCTTGGAACTCTGGCGCTCTGACA
TCCGGCGTGCACACCTTTCCAGCTGTGCTGCAATCCTCCGGCCTG
TACTCTCTGTCCTCCGTCGTGACCGTGCCTTCTAGCTCTCTGGGC
ACCAAGACCTACACCTGTAATGTGGACCACAAGCCTTCCAACACC
AAGGTGGACAAGCGCGTGGAATCTAAGTACGGCCCTCCTTGTCCT
CCATGTCCTGCTCCAGAAGCTGCTGGCGGCCCTTCCGTGTTTCTG
TTCCCTCCAAAGCCTAAGGACACCCTGATGATCTCTCGGACCCCT
GAAGTGACCTGCGTGGTGGTCGATGTGTCCCAAGAGGATCCCGAG
GTGCAGTTCAATTGGTACGTGGACGGCGTGGAAGTGCACAACGCC
AAGACCAAGCCTAGAGAGGAACAGTTCAACTCCACCTACAGAGTG
GTGTCCGTGCTGACAGTGCTGCACCAGGATTGGCTGAACGGCAAA
GAGTACAAGTGCAAGGTGTCCAACAAGGGCCTGCCTTCCAGCATC
GAAAAGACCATCTCCAAGGCCAAGGGCCAGCCTAGGGAACCCCAG
GTTTACACCCTGCCTCCAAGCCAAGAGGAAATGACCAAGAACCAG
GTGTCCCTGACCTGCCTCGTGAAGGGCTTCTACCCTTCCGATATC
GCCGTGGAATGGGAGAGCAATGGCCAGCCAGAGAACAACTACAAG
ACAACCCCTCCTGTGCTGGACTCCGACGGCAGCTTCTTCCTGTAT
TCTCGGCTGACCGTGGACAAGTCCAGATGGCAAGAGGGCAACGTG
TTCTCCTGCTCCGTGATGCACGAGGCCCTGCACAATCACTACACC
CAGAAGTCTCTGTCCCTGAGTCTGGGCAAGTGA

### Fragment

In certain embodiments, an antibody provided herein is an antibody fragment. Antibody fragments include, but are not limited to, Fab, Fab′, Fab′-SH, F(ab′)₂, Fv, and single-chain Fv (scFv) fragments, and other fragments described below, e.g., diabodies, triabodies tetrabodies, and single-domain antibodies. For a review of certain antibody fragments, see Hudson et al., Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (DOMANTIS, Inc., Waltham, Mass.; see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

### Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In another example, a chimeric antibody comprises a human variable region and a non-human constant region (e.g., a constant region derived from a mouse, rat, hamster, rabbit, or non-human primate, such as a monkey). In a further example, a chimeric antibody is a “class switched” antibody in which the class or subclass has been changed from that of the parent antibody. Chimeric antibodies include antigen-binding fragments thereof.

In certain embodiments, an antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain embodiments, an antibody provided herein is a human antibody. Human antibodies can be produced using various techniques known in the art or using techniques described herein or know in the art, such as in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE technology; U.S. Pat. No. 5,770,429 describing HUMAB technology; U.S. Pat. No. 7,041,870 describing K-M MOUSE technology, and U.S. Patent Application Publication No. US 2007/0061900, describing VELOCIMOUSE technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3): 927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al., in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as scFv fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360. Antibodies or antibody fragments isolated from human antibody libraries are considered human antibodies or human antibody fragments herein.

### Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen binding.

### Substitution, Insertion, and Deletion Variants

In certain embodiments, antibody variants having one or more amino acid substitutions are provided. Sites of interest for substitutional mutagenesis include the HVRs and FRs. Conservative substitutions are defined herein. Amino acid substitutions may be introduced into an antibody of interest and the products screened for a desired activity, e.g., retained/improved antigen binding, decreased immunogenicity, or improved ADCC or CDC.

Accordingly, an antibody of the invention can comprise one or more conservative modifications of the CDRs, heavy chain variable region, or light variable regions described herein. A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., one of the amino acid sequences described above). Accordingly, within scope of this invention are heavy chain variable region or light variable regions having one or more point mutations, insertions, deletions, truncations, a fusion protein, or a combination thereof, as well as antibodies having the variant regions.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. (See ncbi.nlm.nih.gov).

As used herein, the term “conservative modifications” refers to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include:
amino acids with basic side chains (e.g., lysine, arginine, histidine),
acidic side chains (e.g., aspartic acid, glutamic acid),
uncharged polar side chains (e.g., glycine, asparagine, glutamine, serine, threonine, tyrosine, cysteine, tryptophan),
nonpolar side chains (e.g., alanine, valine, leucine, isoleucine, proline, phenylalanine, methionine),
beta-branched side chains (e.g., threonine, valine, isoleucine) and
aromatic side chains (e.g., tyrosine, phenylalanine, tryptophan, histidine).

Non-conservative substitutions will entail exchanging a member of one of these classes for another class.

An exemplary substitutional variant is an affinity matured antibody, which may be conveniently generated, e.g., using phage display-based affinity maturation techniques such as those described in e.g., Hoogenboom et al., in Methods in Molecular Biology 178:1-37 (O'Brien et al., ed., Human Press, Totowa, N.J., (2001). Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to another protein (e.g., enzyme) or a polypeptide which increases the serum half-life of the antibody.

### Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 by Co et al. For example, glycosylation of the constant region on N297 may be prevented by mutating the N297 residue to another residue, e.g., N297A, and/or by mutating an adjacent amino acid, e.g., 298 to thereby reduce glycosylation on N297.

Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies described herein to thereby produce an antibody with altered glycosylation. For example, EP 1,176,195 by Hanai et al. describes a cell line with a functionally disrupted FUT8 gene, which encodes a fucosyl transferase, such that antibodies expressed in such a cell line exhibit hypofucosylation. WO 03/035835 by Presta describes a variant Chinese Hamster Ovary cell line, Led 3 cells, with reduced ability to attach fucose to Asn(297)-linked carbohydrates, also resulting in hypofucosylation of antibodies expressed in that host cell (see also Shields, R.L. et al. (2002) J. Biol. Chem. 277:26733-26740). WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180).

### Fc Region Variants

The variable regions of the antibody described herein can be linked (e.g., covalently linked or fused) to an Fc, e.g., an IgG1, IgG2, IgG3 or IgG4 Fc, which may be of any allotype or isoallotype, e.g., for IgG1: Glm, Glm1(a), Glm2(x), Glm3(f), Glm17(z); for IgG2: G2m, G2m23(n); for IgG3: G3m, G3m21(g1), G3m28(g5), G3m11(b0), G3m5(b1), G3m13(b3), G3m14(b4), G3m10(b5), G3m15(s), G3m16(t), G3m6(c3), G3m24(c5), G3m26(u), G3m27(v); and for K: Km, Km1, Km2, Km3 (see, e.g., Jefferies et al. (2009) mAbs 1: 1). In certain embodiments, the antibodies variable regions described herein are linked to an Fc that binds to one or more activating Fc receptors (FcγI, FcγIIa or FcγIIIa), and thereby stimulate ADCC and may cause T cell depletion. In certain embodiments, the antibody variable regions described herein are linked to an Fc that causes depletion.

In certain embodiments, the antibody variable regions described herein may be linked to an Fc comprising one or more modification, typically to alter one or more functional properties of the antibody, such as serum half-life, complement fixation, Fc receptor binding, and/or antigen-dependent cellular cytotoxicity. Furthermore, an antibody described herein may be chemically modified (e.g., one or more chemical moieties can be attached to the antibody) or be modified to alter its glycosylation, to alter one or more functional properties of the antibody. The numbering of residues in the Fc region is that of the EU index of Kabat.

The Fc region encompasses domains derived from the constant region of an immunoglobulin, preferably a human immunoglobulin, including a fragment, analog, variant, mutant or derivative of the constant region. Suitable immunoglobulins include IgG1, IgG2, IgG3, IgG4, and other classes such as IgA, IgD, IgE and IgM. The constant region of an immunoglobulin is defined as a naturally-occurring or synthetically-produced polypeptide homologous to the immunoglobulin C-terminal region, and can include a CH1 domain, a hinge, a CH2 domain, a CH3 domain, or a CH4 domain, separately or in combination. The antibody can have an Fc region from that of IgG (e.g., IgG1, IgG2, IgG3, and IgG4) or other classes such as IgA1, IgA2, IgD, IgE and IgM. The Fc can be a mutant form any of the classes or subclasses.

The constant region of an immunoglobulin is responsible for many important antibody functions including Fc receptor (FcR) binding and complement fixation. There are five major classes of heavy chain constant region, classified as IgA, IgG, IgD, IgE, IgM, each with characteristic effector functions designated by isotype.

Ig molecules interact with multiple classes of cellular receptors. For example, IgG molecules interact with three classes of Fcγ receptors (FcγR) specific for the IgG class of antibody, namely FcγRI, FcγRII, and FcγRIIL. The important sequences for the binding of IgG to the FcγR receptors have been reported to be located in the CH2 and CH3 domains. The serum half-life of an antibody is influenced by the ability of that antibody to bind to an FcR.

In certain embodiments, the Fc region is a variant Fc region, e.g., an Fc sequence that has been modified (e.g., by amino acid substitution, deletion and/or insertion) relative to a parent Fc sequence (e.g., an unmodified Fc polypeptide that is subsequently modified to generate a variant), to provide desirable structural features and/or biological activity. For example, one may make modifications in the Fc region in order to generate an Fc variant that (a) has increased or decreased ADCC, (b) increased or decreased complement-mediated cytotoxicity (CDC), (c) has increased or decreased affinity for Clq and/or (d) has increased or decreased affinity for an Fc receptor relative to the parent Fc. Such Fc region variants will generally comprise at least one amino acid modification in the Fc region. Combining amino acid modifications is thought to be particularly desirable. For example, the variant Fc region may include two, three, four, five, etc. substitutions therein, e.g., of the specific Fc region positions identified herein.

A variant Fc region may also comprise a sequence alteration wherein amino acids involved in disulfide bond formation are removed or replaced with other amino acids. Such removal may avoid reaction with other cysteine-containing proteins present in the host cell used to produce the antibodies described herein. Even when cysteine residues are removed, single chain Fc domains can still form a dimeric Fc domain that is held together non-covalently. In other embodiments, the Fc region may be modified to make it more compatible with a selected host cell. For example, one may remove the PA sequence near the N-terminus of a typical native Fc region, which may be recognized by a digestive enzyme in E. coli such as proline iminopeptidase. In other embodiments, one or more glycosylation sites within the Fc domain may be removed. Residues that are typically glycosylated (e.g., asparagine) may confer cytolytic response. Such residues may be deleted or substituted with unglycosylated residues (e.g., alanine). In other embodiments, sites involved in interaction with complement, such as the Clq binding site, may be removed from the Fc region. For example, one may delete or substitute the EKK sequence of human IgG1. In certain embodiments, sites that affect binding to Fc receptors may be removed, preferably sites other than salvage receptor binding sites. In other embodiments, an Fc region may be modified to remove an ADCC site. ADCC sites are known in the art; see, for example, Molec. Immunol. 29 (5): 633-9 (1992) with regard to ADCC sites in IgGI. Specific examples of variant Fc domains are disclosed for example, in WO 97/34631 and WO 96/32478.

In one embodiment, the hinge region of Fc is modified such that the number of cysteine residues in the hinge region is altered, e.g., increased or decreased. This approach is described further in U.S. Pat. No. 5,677,425 by Bodmer et al. The number of cysteine residues in the hinge region of Fc is altered to, for example, facilitate assembly of the light and heavy chains or to increase or decrease the stability of the antibody. In one embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the CI component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered Clq binding and/or reduced or abolished CDC. This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region may be modified to increase ADCC and/or to increase the affinity for an Fcγ receptor by modifying one or more amino acids at the following positions: 234, 235, 236, 238, 239, 240, 241 , 243, 244, 245, 247, 248, 249, 252, 254, 255, 256, 258, 262, 263, 264, 265, 267, 268, 269, 270, 272, 276, 278, 280, 283, 285, 286, 289, 290, 292, 293, 294, 295, 296, 298, 299, 301, 303, 305, 307, 309, 312, 313, 315, 320, 322, 324, 325, 326, 327, 329, 330, 331, 332, 333, 334, 335, 337, 338, 340, 360, 373, 376, 378, 382, 388, 389, 398, 414, 416, 419, 430, 433, 434, 435, 436, 437, 438 or 439. Exemplary substitutions include 236A, 239D, 239E, 268D, 267E, 268E, 268F, 324T, 332D, and 332E. Exemplary variants include 239D/332E, 236A/332E, 236A/239D/332E, 268F/324T, 267E/268F, 267E/324T, and 267E/268F7324T. Other modifications for enhancing FcγR and complement interactions include but are not limited to substitutions 298A, 333A, 334A, 326A, 247I, 339D, 339Q, 280H, 290S, 298D, 298V, 243L, 292P, 300L, 396L, 305I, and 396L. These and other modifications are reviewed in Strohl, 2009, Current Opinion in Biotechnology 20:685-691.

Fc modifications that increase binding to an Fcγ receptor include amino acid modifications at any one or more of amino acid positions 238, 239, 248, 249, 252, 254, 255, 256, 258, 265, 267, 268, 269, 270, 272, 279, 280, 283, 285, 298, 289, 290, 292, 293, 294, 295, 296, 298, 301, 303, 305, 307, 312, 315, 324, 327, 329, 330, 335, 337, 3338, 340, 360, 373, 376, 379, 382, 388, 389, 398, 414, 416, 419, 430, 434, 435, 437, 438 or 439 of the Fc region, wherein the numbering of the residues in the Fc region is that of the EU index as in abat (WO00/42072).

Other Fc modifications that can be made to Fcs are those for reducing or ablating binding to FcγR and/or complement proteins, thereby reducing or ablating Fc-mediated effector functions such as ADCC, ADCP, and CDC. Exemplary modifications include but are not limited substitutions, insertions, and deletions at positions 234, 235, 236, 237, 267, 269, 325, and 328, wherein numbering is according to the EU index. Exemplary substitutions include but are not limited to 234G, 235G, 236R, 237K, 267R, 269R, 325L, and 328R, wherein numbering is according to the EU index. An Fc variant may comprise 236R/328R. Other modifications for reducing FcγR and complement interactions include substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691.

Optionally, the Fc region may comprise a non-naturally occurring amino acid residue at additional and/or alternative positions known to one skilled in the art (see, e.g., U.S. Pat. Nos. 5,624,821; 6,277,375; 6,737,056; 6,194,551; 7,317,091; 8,101,720; WO00/42072; WO01/58957; WO02/06919; WO04/016750; WO04/029207; WO04/035752; WO04/074455; WO04/099249; WO04/063351; WO05/070963; WO05/040217, WO05/092925 and WO06/020114).

Fc variants that enhance affinity for an inhibitory receptor FcγRIIb may also be used. Such variants may provide an Fc fusion protein with immune-modulatory activities related to FcγRIIb cells, including for example B cells and monocytes. In one embodiment, the Fc variants provide selectively enhanced affinity to FcγRIIb relative to one or more activating receptors. Modifications for altering binding to FcγRIIb include one or more modifications at a position selected from the group consisting of 234, 235, 236, 237, 239, 266, 267, 268, 325, 326, 327, 328, and 332, according to the EU index. Exemplary substitutions for enhancing FcγRIIb affinity include but are not limited to 234D, 234E, 234F, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. Exemplary substitutions include 235Y, 236D, 239D, 266M, 267E, 268D, 268E, 328F, 328W, and 328Y. Other Fc variants for enhancing binding to FcγRllb include 235Y/267E, 236D/267E, 239D/268D, 239D/267E, 267E/268D, 267E/268E, and 267E/328F.

The affinities and binding properties of an Fc region for its ligand may be determined by a variety of in vitro assay methods (biochemical or immunological based assays) known in the art including but not limited to, equilibrium methods (e.g., ELISA, or radioimmunoassay), or kinetics (e.g., BIACORE analysis), and other methods such as indirect binding assays, competitive inhibition assays, fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental immunology, 4th Ed., Lippincott-Raven, Philadelphia (1999), which focuses on antibody-immunogen interactions.

In certain embodiments, the antibody is modified to increase its biological half-life. Various approaches are possible. For example, this may be done by increasing the binding affinity of the Fc region for FcRn. For example, one or more of following residues can be mutated: 252, 254, 256, 433, 435, 436, as described in U.S. Pat. No. 6,277,375. Specific exemplary substitutions include one or more of the following: T252L, T254S, and/or T256F. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. Other exemplary variants that increase binding to FcRn and/or improve pharmacokinetic properties include substitutions at positions 259, 308, 428, and 434, including for example 259I, 308F, 428L, 428M, 434S, 434H, 434F, 434Y, and 434M. Other variants that increase Fc binding to FcRn include: 250E, 250Q, 428L, 428F, 250Q/428L (Hinton et al,, 2004, J. Biol. Chem. 279(8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276(9):6591-6604), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (Dall Acqua et al. Journal of Immunology, 2002, 169:5171-5180, Dall'Acqua et al., 2006, Journal of Biological Chemistry 281:23514-23524). Other modifications for modulating FcRn binding are described in Yeung et al., 2010, J Immunol, 182:7663-7671. In certain embodiments, hybrid IgG isotypes with particular biological characteristics may be used. For example, an IgG1/IgG3 hybrid variant may be constructed by substituting IgG1 positions in the CH2 and/or CH3 region with the amino acids from IgG3 at positions where the two isotypes differ. Thus a hybrid variant IgG antibody may be constructed that comprises one or more substitutions, e.g., 274Q, 276K, 300F, 339T, 356E, 358M, 384S, 392N, 397M, 422I, 435R, and 436F. In other embodiments described herein, an IgG1/IgG2 hybrid variant may be constructed by substituting IgG2 positions in the CH2 and/or CH3 region with amino acids from IgG1 at positions where the two isotypes differ. Thus a hybrid variant IgG antibody may be constructed chat comprises one or more substitutions, e.g., one or more of the following amino acid substitutions: 233E, 234L, 235L, 236G (referring to an insertion of a glycine at position 236), and 321 h.

Moreover, the binding sites on human IgGI for FcγRI, FcγRII, FcγRIII and FcRn have been mapped and variants with improved binding have been described (see Shields, R. L. et al. (2001) J. Biol. Chem. 276:6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A, which has been shown to exhibit enhanced FcγRIIIa binding and ADCC activity (Shields et al., 2001). Other IgG1 variants with strongly enhanced binding to FcγRIIIa have been identified, including variants with S239D/I332E and S239D/I332E/A330L mutations which showed the greatest increase in affinity for FcγRIIIa, a decrease in FcγRIIb binding, and strong cytotoxic activity in cynomolgus monkeys (Lazar et al. , 2006). Introduction of the triple mutations into antibodies such as alemtuzumab (CD52-specific), trastuzumab (HER2/neu-specific), rituximab (CD20-specific), and cetuximab (EGFR-specific) translated into greatly enhanced ADCC activity in vitro, and the S239D/I332E variant showed an enhanced capacity to deplete B cells in monkeys (Lazar et al., 2006). In addition, IgGI mutants containing L235V, F243L, R292P, Y300L and P396L mutations which exhibited enhanced binding to FcγRIIIa and concomitantly enhanced ADCC activity in transgenic mice expressing human FcγRIIIa in models of B cell malignancies and breast cancer have been identified (Stavenhagen et al., 2007; Nordstrom et al., 2011). Other Fc mutants that may be used include: S298A/E333A/L334A, S239D/I332E, S239D/I332E/A330L, L235V/F243L/R292P/Y300L/P396L, and M428L/N434S.

In certain embodiments, an Fc is chosen that has reduced binding to FcγRs. An exemplary Fc, e.g., IgGI Fc, with reduced FcγR binding comprises the following three amino acid substitutions: L234A, L235E and G237A.

In certain embodiments, an Fc is chosen that has reduced complement fixation. An exemplary Fc, e.g., IgG1 Fc, with reduced complement fixation has the following two amino acid substitutions: A330S and P331S.

In certain embodiments, an Fc is chosen that has essentially no effector function, i.e., it has reduced binding to FcγRs and reduced complement fixation. An exemplary Fc, e.g., IgG1 Fc, that is effectorless comprises the following five mutations: L234A, L235E, G237A, A330S and P331S.

When using an IgG4 constant domain, it is usually preferable to include the substitution S228P, which mimics the hinge sequence in IgG1 and thereby stabilizes IgG4 molecules.

### Antibody Derivatives

An antibody provided herein may be further modified to contain additional nonproteinaceous moieties that are known in the art and readily available. The moieties suitable for derivatization of the antibody include but are not limited to water soluble polymers.

Non-limiting examples of water soluble polymers include, but are not limited to, PEG, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed

Another modification of the antibodies described herein is pegylation. An antibody can be pegylated to, for example, increase the biological (e.g., serum) half-life of the antibody. To pegylate an antibody, the antibody, or fragment thereof, typically is reacted with PEG, such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (CI-CIO) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies described herein. See for example, EP 0 154 316 by Nishimura et al. and EP0401384 by Ishikawa et al.

The present invention also encompasses a human monoclonal antibody described herein conjugated to a therapeutic agent, a polymer, a detectable label or enzyme. In one embodiment, the therapeutic agent is a cytotoxic agent. In one embodiment, the polymer is PEG.

### Methods of Productions

Antibodies may be produced using recombinant methods and compositions, e.g., as described in U.S. Pat. No. 4,816,567. In one embodiment, isolated nucleic acid encoding an antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES technology for producing antibodies in transgenic plants).

Vertebrate cells may also be used as hosts. For example, mammalian cell lines that are adapted to grow in suspension may be useful. Other examples of useful mammalian host cell lines are monkey kidney CVI line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include CHO cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### Gene and Cell Therapy

As summarized above, one aspect of this invention includes GIPR inhibition, comprising introducing a GIPR antigen-binding protein in a subject in need thereof. In one embodiment, a cell expressing the antigen-binding protein may be introduced to the subject. In another embodiment, a nucleic acid or nucleic acids encoding the GIPR antigen-binding protein may be introduced into a cell of the subject in a vector such that the nucleic acid(s) remains extrachromosomal or may be integrated into the subject's chromosomal DNA for expression. These methods provide for administering to a subject in need of such treatment a therapeutically effective amount of a nucleic acid or nucleic acids encoding the GIPR antigen-binding protein, or pharmaceutically acceptable composition thereof, for expressing the antigen-binding protein.

The nucleic acid or nucleic acids may or may not be integrated (covalently linked) to chromosomal DNA making up the genome of the subject's target cells. The nucleic acid or nucleic acids may be introduced into the cell such that the nucleic acid remains extrachromosomal. In such a situation, the nucleic acid will be expressed by the cell from the extrachromosomal location. The cells may also be transformed where the exogenous nucleic acid has become integrated into the chromosome so that it is inherited by daughter cells through chromosome replication. The nucleic acid may be introduced into an appropriate vector for extrachromosomal maintenance or for integration into the host. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The gene(s) encoding the antigen-binding protein as described herein may be a polynucleotide or nucleic acid which may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand.

The polynucleotide or nucleic acid compositions or molecules of this invention can include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In vivo expression of transgenes can be carried out by injection of transgenes directly into a specific tissue, such as direct intratracheal, intramuscular or intraarterial injection of naked DNA or of DNA-cationic liposome complexes, or to ex vivo transfection of host cells, with subsequent reinfusion.

Multiple approaches for introducing functional new genetic material into cells, both in vitro and in vivo are known. These approaches include integration of a gene to be expressed into modified retroviruses; integration into non-virus vectors; or delivery of a transgene linked to a heterologous promoter-enhancer element via liposomes; coupled to ligand-specification-based transport systems or the use of naked DNA expression vectors. Direct injection of transgenes into tissue produces localized expression PCT/US90/01515 (Felgner et al.) is directed to methods for delivering a gene coding for a pharmaceutical or immunogenic polypeptide to the interior of a cell of a vertebrate in vivo. While most gene therapy strategies have relied on transgene insertion into retroviral or DNA virus vectors, lipid carriers, may be used to transfect the lung cells of the host.

The polynucleotides or nucleic acids described above may be produced by replication in a suitable host cell. Natural or synthetic polynucleotide fragments coding for a desired fragment can be incorporated into recombinant polynucleotide constructs, usually DNA constructs, capable of introduction into and replication in a prokaryotic or eukaryotic cell. Usually the polynucleotide constructs can be suitable for replication in a unicellular host, such as yeast or bacteria, but may also be intended for introduction to (with and without integration within the genome) cultured mammalian or plant or other eukaryotic cell lines.

The polynucleotides or nucleic acids may also be produced by chemical synthesis and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide or nucleic acid constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native protein or from others or from secreted polypeptides of the same or related species, which allow the protein to cross and/or lodge in cell membranes, and thus attain its functional topology, or be secreted from the cell. Such vectors may be prepared by means of standard recombinant techniques well known in the art.

An appropriate promoter and other necessary vector sequences can be selected so as to be functional in the host, and may include, when appropriate, those naturally associated with immunoglobulin genes. Many useful vectors are known in the art and may be obtained from such vendors as STRATAGENE, NEW ENGLAND BIOLABS, PROMEGA BIOTECH, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Appropriate non-native mammalian promoters might include the early and late promoters from SV40 or promoters derived from murine Moloney leukemia virus, mouse tumor virus, avian sarcoma viruses, adenovirus II, bovine papilloma virus or polyoma. In addition, the construct may be joined to an amplifiable gene so that multiple copies of the gene may be made.

In one embodiment, the nucleic acid construct can include at least one promoter selected from the group consisting of RNA polymerase III, RNA polymerase II, CMV promoter and enhancer, SV40 promoter, an HBV promoter, an HCV promoter, an HSV promoter, an HPV promoter, an EBV promoter, an HTLV promoter, an HIV promoter, and cdc25C promoter, a cyclin a promoter, a cdc2 promoter, a bmyb promoter, a DHFR promoter and an E2F-1 promoter. In some embodiments, one can use an Ubiquitin C promoter for long-term expression.

In accordance with the present invention, there is provided a method of treating a metabolic disorder comprising the administration to a patient in need of such treatment a therapeutically effective amount of a nucleic acid or nucleic acids encoding an antigen-binding protein of this invention, or pharmaceutically acceptable composition thereof. Aspects of the methods include administering to the subject a first nucleic acid alone or in a vector including a coding sequence for antigen-binding protein. Gene therapy methods that utilize the nucleic acid are also provided. Embodiments of the invention include compositions, e.g., nucleic acid alone or in vectors and kits, etc., that find use in the methods.

The methods may lead to increase the expression of the antigen-binding protein when administered to subjects (e.g., mammals). Administration of the vectors to the subject may ameliorate one or more symptoms or markers of the disease or condition.

Any convenient viruses may be utilized in delivering the vector of interest to the subject. Viruses of interest include, but are not limited to a retrovirus, an adenovirus, an adeno-associated virus (AAV), a herpes simplex virus and a lentivirus. Viral gene therapy vectors are well known in the art, see e.g., Heilbronn & Weger (2010) Handb Exp Pharmacal. 197:143-70. Vectors of interest include integrative and non-integrative vectors such as those based on retroviruses, adenoviruses (AdV), adeno-associated viruses (AAV), lentiviruses, pox viruses, alphaviruses, and herpes viruses.

In some cases, non-integrative viral vectors, such as AAV, may be utilized. In one aspect, non-integrative vectors do not cause any permanent genetic modification. The vectors may be targeted to adult tissues to avoid having the subjects under the effect of constitutive expression from early stages of development. In some instances, non-integrative vectors effectively incorporate a safety mechanism to avoid over-proliferation of MRCKα and/or NKA β1 expressing cells. The cells may lose the vector (and, as a consequence, the protein expression) if they start proliferating quickly.

Non-integrative vectors of interest include those based on adenoviruses (AdV) such as gutless adenoviruses, adeno-associated viruses (AAV), integrase deficient lentiviruses, pox viruses, alphaviruses, and herpes viruses. In certain embodiments, the non-integrative vector used in the invention is an adeno-associated virus-based non-integrative vector, similar to natural adeno-associated virus particles. Examples of adeno-associated virus-based non integrative vectors include vectors based on any AAV serotype, i.e., AAV1, AAV2, AAV3, AAV4, AAV5, AAV6, AAV7, AAV8, AAV9, AAV10, AAV11 and pseudotyped AAV. Vectors of interest include those capable of transducing a broad range of tissues at high efficiency, with poor immunogenicity and an excellent safety profile. In some cases, the vectors transduce post-mitotic cells and can sustain long-term gene expression (up to several years) both in small and large animal models of the related disorders.

### Compositions and Formulations

The antigen binding proteins (including antibodies) of this invention represent an excellent way for the development of therapies either alone or in combination with additional agents for the treatment of a metabolic disorder.

Pharmaceutical compositions that comprise a GIPR antigen binding protein are also provided and can be utilized in any of the preventive and therapeutic methods disclosed herein. In an embodiment, a therapeutically effective amount of one or a plurality of the antigen binding proteins and a pharmaceutically acceptable diluent, carrier, solubilizer, emulsifier, preservative, and/or adjuvant are also provided. Acceptable formulation materials are nontoxic to recipients at the dosages and concentrations employed.

In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. REMINGTON'S PHARMACEUTICAL SCIENCES, 18″ Edition, (A. R. Genrmo, ed.), 1990, Mack Publishing Company provides additional details and options for suitable agents that can be incorporated into the pharmaceutical compositions.

In certain embodiments, the optimal pharmaceutical composition will be determined by one skilled in the art depending upon, for example, the intended route of administration, delivery format and desired dosage. See, for example, REMINGTON'S PHARMACEUTICAL SCIENCES, supra. In certain embodiments, such compositions may influence the physical state, stability, rate of in vivo release and rate of in vivo clearance of the antigen binding proteins disclosed. In certain embodiments, the primary vehicle or carrier in a pharmaceutical composition may be either aqueous or non-aqueous in nature. For example, a suitable vehicle or carrier may be water for injection or physiological saline solution. In certain embodiments, GIPR antigen binding protein compositions may be prepared for storage by mixing the selected composition having the desired degree of purity with optional formulation agents (REMINGTON'S PHARMACEUTICAL SCIENCES, supra) in the form of a lyophilized cake or an aqueous solution. Further, in certain embodiments, the GIPR antigen binding protein may be formulated as a lyophilizate using appropriate excipients such as sucrose.

The pharmaceutical compositions can be selected for parenteral delivery. Alternatively, the compositions may be selected for inhalation or for delivery through the digestive tract, such as orally. Preparation of such pharmaceutically acceptable compositions is within the skill of the art.

The formulation components are present preferably in concentrations that are acceptable to the site of administration. In certain embodiments, buffers are used to maintain the composition at physiological pH or at a slightly lower pH, typically within a pH range of from about 5 to about 8.

When parenteral administration is contemplated, the therapeutic compositions may be provided in the form of a pyrogen-free, parenterally acceptable aqueous solution comprising the desired human GIPR antigen binding protein in a pharmaceutically acceptable vehicle. A particularly suitable vehicle for parenteral injection is sterile distilled water in which the GIPR antigen binding protein is formulated as a sterile, isotonic solution, properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that may provide controlled or sustained release of the product which can be delivered via depot injection. In certain embodiments, hyaluronic acid may also be used, having the effect of promoting sustained duration in the circulation. In certain embodiments, implantable drug delivery devices may be used to introduce the desired antigen binding protein.

Certain pharmaceutical compositions are formulated for inhalation. In some embodiments, GIPR antigen binding proteins are formulated as a dry, inhalable powder. In specific embodiments, GIPR antigen binding protein inhalation solutions may also be formulated with a propellant for aerosol delivery. In certain embodiments, solutions may be nebulized. Pulmonary administration and formulation methods therefore are further described in International Patent Application No. PCT/US94/001875, which is incorporated by reference and describes pulmonary delivery of chemically modified proteins. Some formulations can be administered orally. GIPR antigen binding proteins that are administered in this fashion can be formulated with or without carriers customarily used in the compounding of solid dosage forms such as tablets and capsules. In certain embodiments, a capsule may be designed to release the active portion of the formulation at the point in the gastrointestinal tract when bioavailability is maximized and pre-systemic degradation is minimized. Additional agents can be included to facilitate absorption of the GIPR antigen binding protein. Diluents, flavorings, low melting point waxes, vegetable oils, lubricants, suspending agents, tablet disintegrating agents, and binders may also be employed.

Additional pharmaceutical compositions will be evident to those skilled in the art, including formulations involving GIPR binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. See, for example, International Patent Application No. PCT/US93/00829, which is incorporated by reference and describes controlled release of porous polymeric microparticles for delivery of pharmaceutical compositions. Sustained-release preparations may include semipermeable polymer matrices in the form of shaped articles, e.g., films, or microcapsules. Sustained release matrices may include polyesters, hydrogels, polylactides (as disclosed in U.S. Pat. No. 3,773,919 and European Patent Application Publication No. EP 058481, each of which is incorporated by reference), copolymers of L-glutamic acid and gamma ethyl-L-glutamate (Sidman et al., 1983, Biopolymers 2:547-556), poly (2-hydroxyethyl-inethacrylate) (Langer et al., 1981, J. Biomed. Mater. Res. 15:167-277 and Langer, 1982, Chem. Tech. 12:98-105), ethylene vinyl acetate (Langer et al., 1981, supra) or poly-D(-)-3-hydroxybutyric acid (European Patent Application Publication No. EP 133,988). Sustained release compositions may also include liposomes that can be prepared by any of several methods known in the art. See, e.g., Eppstein et al., 1985, Proc. Natl. Acad. Sci. U.S.A. 82:3688-3692; European Patent Application Publication Nos. EP 036,676; EP 088,046 and EP 143,949, incorporated by reference.

Pharmaceutical compositions used for in vivo administration are typically provided as sterile preparations. Sterilization can be accomplished by filtration through sterile filtration membranes. When the composition is lyophilized, sterilization using this method may be conducted either prior to or following lyophilization and reconstitution. Compositions for parenteral administration can be stored in lyophilized form or in a solution. Parenteral compositions generally are placed into a container having a sterile access port, for example, an intravenous solution bag or vial having a stopper pierceable by a hypodermic injection needle.

In certain formulations, an antigen binding protein has a concentration of at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL or 150 mg/mL. In one embodiment, a pharmaceutical composition comprises the antigen binding protein, a buffer and polysorbate. In other embodiments, the pharmaceutical composition comprises an antigen binding protein, a buffer, sucrose and polysorbate. An example of a pharmaceutical composition is one containing 50-100 mg/mL of antigen binding protein, 5-20 mM sodium acetate, 5-10% w/v sucrose, and 0.002-0.008% w/v polysorbate. Certain, compositions, for instance, contain 65-75 mg/mL of an antigen binding protein in 9-11 mM sodium acetate buffer, 8-10% w/v sucrose, and 0.005-0.006% w/v polysorbate. The pH of certain such formulations is in the range of 4.5-6. Other formulations have a pH of 5.0-5.5 (e.g., pH of 5.0, 5.2 or 5.4).

Once the pharmaceutical composition has been formulated, it may be stored in sterile vials as a solution, suspension, gel, emulsion, solid, crystal, or as a dehydrated or lyophilized powder. Such formulations may be stored either in a ready-to-use form or in a form (e.g., lyophilized) that is reconstituted prior to administration. Kits for producing a single-dose administration unit are also provided. Certain kits contain a first container having a dried protein and a second container having an aqueous formulation. In certain embodiments, kits containing single and multi-chambered pre-filled syringes (e.g., liquid syringes and lyosyringes) are provided. The therapeutically effective amount of a GIPR antigen binding protein-containing pharmaceutical composition to be employed will depend, for example, upon the therapeutic context and objectives. One skilled in the art will appreciate that the appropriate dosage levels for treatment will vary depending, in part, upon the molecule delivered, the indication for which the GIPR antigen binding protein is being used, the route of administration, and the size (body weight, body surface or organ size) and/or condition (the age and general health) of the patient. In certain embodiments, the clinician may titer the dosage and modify the route of administration to obtain the optimal therapeutic effect.

Dosing frequency will depend upon the pharmacokinetic parameters of the particular GIPR antigen binding protein in the formulation used. Typically, a clinician administers the composition until a dosage is reached that achieves the desired effect. The composition may therefore be administered as a single dose, or as two or more doses (which may or may not contain the same amount of the desired molecule) over time, or as a continuous infusion via an implantation device or catheter. Appropriate dosages may be ascertained through use of appropriate dose-response data. In certain embodiments, the antigen binding proteins can be administered to patients throughout an extended time period. In certain embodiments, the antigen binding protein is dosed every two weeks, every month, every two months, every three months, every four months, every five months, or every six months.

The route of administration of the pharmaceutical composition is in accord with known methods, e.g., orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions may be administered by bolus injection or continuously by infusion, or by implantation device.

The composition also may be administered locally via implantation of a membrane, sponge or another appropriate material onto which the desired molecule has been absorbed or encapsulated. In certain embodiments, where an implantation device is used, the device may be implanted into any suitable tissue or organ, and delivery of the desired molecule may be via diffusion, timed-release bolus, or continuous administration.

It also may be desirable to use GIPR antigen binding protein pharmaceutical compositions according to the disclosed ex vivo. In such instances, cells, tissues or organs that have been removed from the patient are exposed to GIPR antigen binding protein pharmaceutical compositions after which the cells, tissues and/or organs are subsequently implanted back into the patient.

A physician will be able to select an appropriate treatment indication and target lipid levels depending on the individual profile of a particular patient. One well-accepted standard for guiding treatment of hyperlipidemia is the Third Report of the National Cholesterol Education Program (NCEP) Expert Panel on Detection, Evaluation, and Treatment of the High Blood Cholesterol in Adults (Adult Treatment Panel III) Final Report, National Institutes of Health, NIH Publication No. 02-5215 (2002), the printed publication of which is hereby incorporated by reference in its entirety.

The efficacy of a particular dose can be assessed by reference to biomarkers or improvement in certain physiological parameters. Examples of suitable biomarkers and parameters include those described in the examples below, such as one or more of glucose, cholesterol, triglyceride, liver enzymes (e.g., ALT and AST), lipid profile, liver biomarkers (e.g., HCY and hYP), as well as the ratio of free cholesterol to plasma lipid, free cholesterol to membrane protein, phospatidylcholine to sphingomyelin, or HDL-C levels.

Also provided herein are compositions comprising a GIPR antigen binding protein and one or more additional therapeutic agents, as well as methods in which such agents are administered concurrently or sequentially with a GIPR antigen binding protein for use in the preventive and therapeutic methods disclosed herein. The one or more additional agents can be co-formulated with a GIPR antigen binding protein or can be co-administered with a GIPR antigen binding protein. In general, the therapeutic methods, compositions and compounds may also be employed in combination with other therapeutics in the treatment of various disease states, with the additional agents being administered concurrently.

### GLP-1 Receptor Agonist

In one aspect the present invention is directed to a method of treating a subject with a metabolic disorder, the method comprising administering to the subject a therapeutically effective amount of a GLP-1 receptor agonist and a therapeutically effective amount of a GIPR antagonist that specifically binds to a protein having an amino acid sequence having at least 90% amino acid sequence identity to an amino acid sequence of a GIPR.

A “GLP-1 receptor agonist” or “GLP-1 agonist” refers to compounds having GLP-1 receptor activity. Such exemplary compounds include exendins, exendin analogs, exendin agonists, GLP-1(7-37), GLP-1(7-37) analogs, GLP-1(7-37) agonists, and the like. The GLP-1 receptor agonist compounds may optionally be amidated. The terms “GLP-1 receptor agonist” and “GLP-1 receptor agonist compound” have the same meaning. Various GLP-1 receptor agonists, such as exendins, exendin analogs, GLP-1(7-37), GLP-1(7-37) analogs, GLP-1(7-37) agonists, are known in the art and described in, e.g., US20170275370, the content of which is incorporated by reference in its entirety.

The term “exendin” includes naturally occurring (or synthetic versions of naturally occurring) exendin peptides that are found in the salivary secretions of the Gila monster. Exendins of particular interest include exendin-3 and exendin-4. The exendins, exendin analogs, and exendin agonists for use in the methods described herein may optionally be amidated, and may also be in an acid form, pharmaceutically acceptable salt form, or any other physiologically active form of the molecule.

In one embodiment, the molar ratio of a GLP-1 receptor agonist to a GIPR antagonist is from about 1:1 to 1:110, 1:1 to 1:100, 1:1 to 1:75, 1:1 to 1:50, 1:1 to 1:25, 1:1 to 1:10, 1:1 to 1:5, and 1:1. In one embodiment, the molar ratio of a GIPR antagonist to a GLP-1 receptor agonist is from about 1:1 to 1:110, 1:1 to 1:100, 1:1 to 1:75, 1:1 to 1:50, 1:1 to 1:25, 1:1 to 1:10, and 1:1 to 1:5. In one embodiment, the GLP-1 receptor agonist is used in combination with the GIPR antagonist at therapeutically effective molar ratios of between about 1:1.5 to 1:150, preferably 1:2 to 1:50. In one embodiment, the GLP-1 receptor agonist and the GIPR antagonist are present in doses that are at least about 1.1 to 1.4, 1.5, 2, 3, 4, 5, 6, 7, 8, 9, or 10 fold lower than the doses of each compound alone required to treat a condition and/or disease.

Pharmaceutical compositions containing the GLP-1 receptor agonist compounds described herein may be provided for peripheral administration, such as parenteral (e.g., subcutaneous, intravenous, intramuscular), a continuous infusion (e.g., intravenous drip, intravenous bolus, intravenous infusion), topical, nasal, or oral administration. Suitable pharmaceutically acceptable carriers and their formulation are described in standard formulation treatises, such as Remington's Pharmaceutical Sciences by Martin; and Wang et al., Journal of Parenteral Science and Technology, Technical Report No. 10, Supp. 42:2S (1988). The GLP-1 receptor agonist compounds described herein can be provided in parenteral compositions for injection or infusion. They can, for example, be suspended in water; an inert oil, such as a vegetable oil (e.g., sesame, peanut, olive oil, and the like); or other pharmaceutically acceptable carrier. In one embodiment, the compounds are suspended in an aqueous carrier, for example, in an isotonic buffer solution at a pH of about 3.0 to 8.0, or about 3.0 to 5.0. The compositions may be sterilized by conventional sterilization techniques or may be sterile filtered. The compositions may contain pharmaceutically acceptable auxiliary substances as required to approximate physiological conditions, such as pH buffering agents.

Useful buffers include for example, acetic acid buffers. A form of repository or “depot” slow release preparation may be used so that therapeutically effective amounts of the preparation are delivered into the bloodstream over many hours or days following subcutaneous injection, transdermal injection or other delivery method. The desired isotonicity may be accomplished using sodium chloride or other pharmaceutically acceptable agents such as dextrose, boric acid, sodium tartrate, propylene glycol, polyols (such as mannitol and sorbitol), or other inorganic or organic solutes. In one embodiment for intravenous infusion, the formulation may comprise (i) the GLP-1 receptor agonist compound, (2) sterile water, and, optionally (3) sodium chloride, dextrose, or a combination thereof

Carriers or excipients can also be used to facilitate administration of the GLP-1 receptor agonist compounds. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents.

The GLP-1 receptor agonist compounds can also be formulated as pharmaceutically acceptable salts (e.g., acid addition salts) and/or complexes thereof. Pharmaceutically acceptable salts are non-toxic salts at the concentration at which they are administered. Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid. Such salts may be prepared by, for example, reacting the free acid or base forms of the product with one or more equivalents of the appropriate base or acid in a solvent or medium in which the salt is insoluble, or in a solvent such as water which is then removed in vacuo or by freeze-drying or by exchanging the ions of an existing salt for another ion on a suitable ion exchange resin.

Exemplary pharmaceutical formulations of GLP-1 receptor agonist compounds are described in U.S. Pat. Nos. 7,521,423, 7,456,254; US 20170275370, US20040106547, WO 2006/068910, WO 2006/125763, and the like, the disclosures of which are incorporated by reference herein.

The therapeutically effective amount of the GLP-1 receptor agonist compounds described herein for use in the methods described herein will typically be from about 0.01 μg to about 5 mg; about 0.1μ to about 2.5 mg; about 1 μg to about 1 mg; about 1 μg to about 50 μg; or about 1 μg to about 25 μg. Alternatively, the therapeutically effective amount of the GLP-1 receptor agonist compounds may be from about 0.001 μg to about 100 μg based on the weight of a 70 kg patient; or from about 0.01 μg to about 50 μg based on the weight of a 70 kg patient. These therapeutically effective doses may be administered once/day, twice/day, thrice/day, once/week, biweekly, or once/month, depending on the formulation. The exact dose to be administered is determined, for example, by the formulation, such as an immediate release formulation or an extended release formulation. For transdermal, nasal or oral dosage forms, the dosage may be increased from about 5-fold to about 10-fold.

In certain embodiments the GLP-1 receptor agonist will be administered concurrently with the GIPR antigen binding protein. In one embodiment the GLP-1 receptor agonist will be administered after the GIPR antigen binding protein. In one embodiment the GLP-1 receptor agonist will be administered before the GIPR antigen binding protein. In certain embodiments the subject or patient will already be being treated with a GLP-1 receptor agonist before being subjected to further treatment with a GIPR antigen binding protein.

### Uses and Methods

The antigen binding proteins disclosed herein have a variety of utilities. The antigen binding proteins, for instance, are useful in specific binding assays, affinity purification of GIPR, and in screening assays to identify other antagonists of GIPR activity. Other uses for the antigen binding proteins include, for example, diagnosis of GIPR-associated diseases or conditions and screening assays to determine the presence or absence of GIPR. Given that the antigen binding proteins that are provided are antagonists, the GIPR antigen binding proteins have value in therapeutic methods to reduce weight gain, even while maintaining or increasing food intake, increasing % fat mass and increasing % lean mass, improving glucose tolerance, decreasing insulin levels, decreasing cholesterol and triglyceride levels. Accordingly, the antigen binding proteins have utility in the treatment and prevention of diabetes, e.g., type 2 diabetes, obesity, dyslipidemia, elevated glucose levels or elevated insulin levels.

### Treatment Methods

The current treatments for diseases and/or conditions associated with GIPR are sub-optimal. GIPR antigen binding protein, and in particular anti-GIPR antibodies, are promising in treating these diseases or conditions in part due to their high degree of specificity, limited off-target effects, and superb safety profile. The GIPR antigen binding protein, antibodies, compositions and formulations described herein can be used to inhibit GIPR and thereby treating the diseases or conditions. Accordingly, the GIPR binding proteins, e.g., antibodies, described herein can be used to can be used to treat, diagnose or ameliorate, a metabolic condition or disorder.

In one aspect, the GIPR binding proteins, e.g., antibodies, described herein can be used to can be used to treat or ameliorate a metabolic condition or disorder. In one embodiment, the metabolic disorder to be treated is a fatty liver disease. In one embodiment, the metabolic disorder to be treated is diabetes, e.g., type 2 diabetes. In another embodiment, the metabolic condition or disorder is obesity. In other embodiments the metabolic condition or disorder is dyslipidemia, elevated glucose levels, elevated insulin levels or diabetic nephropathy. For example, a metabolic condition or disorder that can be treated or ameliorated using a GIPR binding peptide includes a state in which a human subject has a fasting blood glucose level of 125 mg/dL or greater, for example 130, 135, 140, 145, 150, 155, 160, 165, 170, 175, 180, 185, 190, 195, 200 or greater than 200 mg/dL. Blood glucose levels can be determined in the fed or fasted state, or at random. The metabolic condition or disorder can also comprise a condition in which a subject is at increased risk of developing a metabolic condition. For a human subject, such conditions include a fasting blood glucose level of 100 mg/dL. Conditions that can be treated using a pharmaceutical composition comprising a GIPR binding protein can also be found in the American Diabetes Association Standards of Medical Care in Diabetes Care-2011, American Diabetes Association, Diabetes Care Vol. 34, No. Supplement 1, S11-S61, 2010, incorporated herein by reference.

In application, a metabolic disorder or condition, such as a fatty liver disease, Type 2 diabetes, elevated glucose levels, elevated insulin levels, dyslipidemia, obesity or diabetic nephropathy, can be treated by administering a therapeutically effective dose of a GIPR binding protein to a patient in need thereof. The administration can be performed as described herein, such as by IV injection, intraperitoneal (IP) injection, subcutaneous injection, intramuscular injection, or orally in the form of a tablet or liquid formation. In some situations, a therapeutically effective or preferred dose of a GIPR binding protein can be determined by a clinician. A therapeutically effective dose of GIPR binding protein will depend, inter alia, upon the administration schedule, the unit dose of agent administered, whether the GIPR binding protein is administered in combination with other therapeutic agents, the immune status and the health of the recipient. The term “therapeutically effective dose,” as used herein, means an amount of GIPR binding protein that elicits a biological or medicinal response in a tissue system, animal, or human being sought by a researcher, medical doctor, or other clinician, which includes alleviation or amelioration of the symptoms of the disease or disorder being treated, i.e., an amount of a GIPR binding protein that supports an observable level of one or more desired biological or medicinal response, for example lowering blood glucose, insulin, triglyceride, or cholesterol levels; reducing body weight; or improving glucose tolerance, energy expenditure, or insulin sensitivity.

It is noted that a therapeutically effective dose of a GIPR binding protein can also vary with the desired result. Thus, for example, in situations in which a lower level of blood glucose is indicated a dose of GIPR binding protein will be correspondingly higher than a dose in which a comparatively lower level of blood glucose is desired. Conversely, in situations in which a higher level of blood glucose is indicated a dose of GIPR binding protein will be correspondingly lower than a dose in which a comparatively higher level of blood glucose is desired. In various embodiments, a subject is a human having a blood glucose level of 100 mg/dL or greater can be treated with a GIPR binding protein.

In one embodiment, a method of the instant disclosure comprises first measuring a baseline level of one or more metabolically-relevant compounds such as glucose, insulin, cholesterol, lipid in a subject. A pharmaceutical composition comprising a GIPR binding protein is then administered to the subject. After a desired period of time, the level of the one or more metabolically-relevant compounds (e.g., blood glucose, insulin, cholesterol, lipid) in the subject is again measured. The two levels can then be compared in order to determine the relative change in the metabolically-relevant compound in the subject. Depending on the outcome of that comparison another dose of the pharmaceutical composition comprising a GIPR binding protein can be administered to achieve a desired level of one or more metabolically-relevant compound.

As noted above, a pharmaceutical composition comprising a GIPR binding protein can be co-administered with another compound. The identity and properties of compound co-administered with the GIPR binding protein will depend on the nature of the condition to be treated or ameliorated. A non-limiting list of examples of compounds that can be administered in combination with a pharmaceutical composition comprising a GIPR binding protein include rosiglitizone, pioglitizone, repaglinide, nateglitinide, metformin, exenatide, stiagliptin, pramlintide, glipizide, glimeprirideacarbose, and miglitol.

### Diagnostic

The GIPR antigen binding proteins that are provided herein are useful for detecting GIPR in biological samples. For instance, the GIPR antigen binding proteins can be used in diagnostic assays, e.g., binding assays to detect and/or quantify GIPR expressed in a sample, such as serum.

The antigen binding proteins of the described can be used for diagnostic purposes to detect, diagnose, or monitor diseases and/or conditions associated with GIPR. The disclosed antigen binding proteins provide a means for the detection of the presence of GIPR in a sample using classical immunohistological methods known to those of skill in the art (e.g., Tijssen, 1993, Practice and Theory of Enzyme Immunoassays, Vol 15 (Eds R. H. Burdon and P. H. van Knippenberg, Elsevier, Amsterdam); Zola, 1987, Monoclonal Antibodies: A Manual of Techniques, pp. 147-158 (CRC Press, Inc.); Jalkanen et al., 1985, J. Cell. Biol. 101:976-985; Jalkanen et al., 1987, J. Cell Biol. 105:3087-3096). The detection of GIPR can be performed in vivo or in vitro.

Diagnostic applications provided herein include use of the antigen binding proteins to detect expression of GIPR. Examples of methods useful in the detection of the presence of GIPR include immunoassays, such as the enzyme linked immunosorbent assay (ELISA) and the radioimmunoassay (RIA).

For diagnostic applications, the antigen binding protein typically will be labeled with a detectable labeling group. Suitable labeling groups include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, and ¹³¹I), fluorescent groups (e.g., FITC, rhodamine, lanthanide phosphors), enzymatic groups (e.g., horseradish peroxidase, luciferase, .beta.-galactosidase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the antigen binding protein via spacer arms of various lengths to reduce potential steric hindrance. Various methods for labeling proteins are known in the art and may be used.

In some embodiments, the GIPR antigen binding protein is isolated and measured using techniques known in the art. See, for example, Harlow and Lane, 1988, Antibodies: A Laboratory Manual, New York: Cold Spring Harbor (ed. 1991 and periodic supplements); John E. Coligan, ed., 1993, Current Protocols In Immunology New York: John Wiley & Sons.

Another aspect of the disclosed provides for detecting the presence of a test molecule that competes for binding to GIPR with the antigen binding proteins provided. An example of one such assay would involve detecting the amount of free antigen binding protein in a solution containing an amount of GIPR in the presence or absence of the test molecule. An increase in the amount of free antigen binding protein (i.e., the antigen binding protein not bound to GIPR) would indicate that the test molecule is capable of competing for GIPR binding with the antigen binding protein. In one embodiment, the antigen binding protein is labeled with a labeling group. Alternatively, the test molecule is labeled and the amount of free test molecule is monitored in the presence and absence of an antigen binding protein.

### Kit

Also provided are kits for practicing the disclosed methods. Such kits can comprise a pharmaceutical composition such as those described herein, including nucleic acids encoding the peptides or proteins provided herein, vectors and cells comprising such nucleic acids, and pharmaceutical compositions comprising such nucleic acid-containing compounds, which can be provided in a sterile container. Optionally, instructions on how to employ the provided pharmaceutical composition in the treatment of a metabolic disorder can also be included or be made available to a patient or a medical service provider.

In one aspect, a kit comprises (a) a pharmaceutical composition comprising a therapeutically effective amount of a GIPR binding protein; and (b) one or more containers for the pharmaceutical composition. Such a kit can also comprise instructions for the use thereof; the instructions can be tailored to the precise metabolic disorder being treated. The instructions can describe the use and nature of the materials provided in the kit. In certain embodiments, kits include instructions for a patient to carry out administration to treat a metabolic disorder, such as a fatty liver disease, elevated glucose levels, elevated insulin levels, obesity, type 2 diabetes, dyslipidemia or diabetic nephropathy.

Instructions can be printed on a substrate, such as paper or plastic, etc., and can be present in the kits as a package insert, in the labeling of the container of the kit or components thereof (e.g., associated with the packaging), etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, such as over the internet, are provided. An example of this embodiment is a kit that includes a web address where the instructions can be viewed and/or from which the instructions can be downloaded.

Often it will be desirable that some or all components of a kit are packaged in suitable packaging to maintain sterility. The components of a kit can be packaged in a kit containment element to make a single, easily handled unit, where the kit containment element, e.g., box or analogous structure, may or may not be an airtight container, e.g., to further preserve the sterility of some or all of the components of the kit.

### Definitions

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. The modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The term “oligonucleotide” means a polynucleotide comprising 200 or fewer nucleotides. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

The term “isolated nucleic acid molecule” refers to a single or double-stranded polymer of deoxyribonucleotide or ribonucleotide bases read from the 5′ to the 3′ end (e.g., a GIPR nucleic acid sequence provided herein), or an analog thereof, that has been separated from at least about 50 percent of polypeptides, peptides, lipids, carbohydrates, polynucleotides or other materials with which the nucleic acid is naturally found when total nucleic acid is isolated from the source cells. Preferably, an isolated nucleic acid molecule is substantially free from any other contaminating nucleic acid molecules or other molecules that are found in the natural environment of the nucleic acid that would interfere with its use in polypeptide production or its therapeutic, diagnostic, prophylactic or research use.

The term “vector” means any molecule or entity (e.g., nucleic acid, plasmid, bacteriophage or virus) used to transfer protein coding information into a host cell. In some embodiments, a “vector” refers to a delivery vehicle that (a) promotes the expression of a polypeptide-encoding nucleic acid sequence; (b) promotes the production of the polypeptide therefrom; (c) promotes the transfection/transformation of target cells therewith; (d) promotes the replication of the nucleic acid sequence; (e) promotes stability of the nucleic acid; (f) promotes detection of the nucleic acid and/or transformed/transfected cells; and/or (g) otherwise imparts advantageous biological and/or physiochemical function to the polypeptide-encoding nucleic acid. A vector can be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors.

The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

As used herein, “operably linked” means that the components to which the term is applied are in a relationship that allows them to carry out their inherent functions under suitable conditions. For example, a control sequence in a vector that is “operably linked” to a protein coding sequence is ligated thereto so that expression of the protein coding sequence is achieved under conditions compatible with the transcriptional activity of the control sequences.

The term “host cell” means a cell that has been transformed with a nucleic acid sequence and thereby expresses a gene of interest. The term includes the progeny of the parent cell, whether or not the progeny is identical in morphology or in genetic make-up to the original parent cell, so long as the gene of interest is present.

The terms “polypeptide” or “protein” are used interchangeably herein to refer to a polymer of amino acid residues. The terms also apply to amino acid polymers in which one or more amino acid residues is an analog or mimetic of a corresponding naturally occurring amino acid, as well as to naturally occurring amino acid polymers. The terms can also encompass amino acid polymers that have been modified, e.g., by the addition of carbohydrate residues to form glycoproteins, or phosphorylated. Polypeptides and proteins can be produced by a naturally-occurring and non-recombinant cell; or it is produced by a genetically-engineered or recombinant cell, and comprise molecules having the amino acid sequence of the native protein, or molecules having deletions from, additions to, and/or substitutions of one or more amino acids of the native sequence. The terms “polypeptide” and “protein” specifically encompass GIPR antigen binding proteins, antibodies, or sequences that have deletions from, additions to, and/or substitutions of one or more amino acids of an antigen-binding protein. The term “polypeptide fragment” refers to a polypeptide that has an amino-terminal deletion, a carboxyl-terminal deletion, and/or an internal deletion as compared with the full-length protein. Such fragments may also contain modified amino acids as compared with the full-length protein. In certain embodiments, fragments are about five to 500 amino acids long. For example, fragments may be at least 5, 6, 8, 10, 14, 20, 50, 70, 100, 110, 150, 200, 250, 300, 350, 400, or 450 amino acids long. Useful polypeptide fragments include immunologically functional fragments of antibodies, including binding domains.

The term “isolated polypeptide” refers to a polypeptide (e.g., a GIPR polypeptide sequence provided herein or an antigen binding protein of the present invention) that has been separated from at least about 50 percent of polypeptides, peptides, lipids, carbohydrates, polynucleotides, or other materials with which the polypeptide is naturally found when isolated from a source cell. Preferably, the isolated polypeptide is substantially free from any other contaminating polypeptides or other contaminants that are found in its natural environment that would interfere with its therapeutic, diagnostic, prophylactic or research use.

The term “encoding” refers to a polynucleotide sequence encoding one or more amino acids. The term does not require a start or stop codon.

A “variant” of a polypeptide (e.g., an antigen binding protein such as an antibody) comprises an amino acid sequence wherein one or more amino acid residues are inserted into, deleted from and/or substituted into the amino acid sequence relative to another polypeptide sequence. Variants include fusion proteins.

A functional variant or equivalent of a reference peptide, polypeptide, or protein refers to a polypeptide derivative of the reference peptide, polypeptide, or protein, e.g., a protein having one or more point mutations, insertions, deletions, truncations, a fusion protein, or a combination thereof. It retains substantially the activity to of the reference peptide, polypeptide, or protein. In general, the functional equivalent is at least 60% (e.g., any number between 60% and 100%, inclusive, e.g., 60%, 70%, 80%, 85%, 90%, 95%, and 99%) identical to the reference peptide, polypeptide, or protein.

A “derivative” of a polypeptide is a polypeptide (e.g., an antigen binding protein such as an antibody) that has been chemically modified in some manner distinct from insertion, deletion, or substitution variants, e.g., via conjugation to another chemical moiety.

The term “antibody” as referred to herein includes whole antibodies and any antigen-binding fragment or single chains thereof. Whole antibodies are glycoproteins comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, C_{H}1, C_{H}2 and C_{H}3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The heavy chain variable region CDRs and FRs are HFR1, HCDR1, HFR2, HCDR2, HFR3, HCDR3, HFR4. The light chain variable region CDRs and FRs are LFR1, LCDR1, LFR2, LCDR2, LFR3, LCDR3, LFR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (CIq) of the classical complement system.

The term “antigen-binding fragment or portion” of an antibody (or simply “antibody fragment or portion”), as used herein, refers to one or more fragments of an antibody that retain the ability to specifically bind to an antigen (e.g., GIPR). It has been shown that the antigen-binding function of an antibody can be performed by fragments of a full-length antibody. Examples of binding fragments encompassed within the term “antigen-binding fragment or portion” of an antibody include (i) a Fab fragment, a monovalent fragment consisting of the V_{L}, V_{H}, C_{L} and C_{H}I domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3^{rd} ed. 1993)); (iv) a Fd fragment consisting of the V_{H} and C_{H}I domains; (v) a Fv fragment consisting of the V_{L} and VH domains of a single arm of an antibody, (vi) a dAb fragment (Ward et al., (1989) Nature 341:544-546), which consists of a VH domain; (vii) an isolated CDR; and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv or scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment or portion” of an antibody. These antibody fragments are obtained using conventional techniques known to those with skill in the art, and the fragments are screened for utility in the same manner as are intact antibodies.

An “isolated antibody”, as used herein, is intended to refer to an antibody that is substantially free of other antibodies having different antigenic specificities (e.g., an isolated antibody that specifically binds to a specific antigen, such as GIPR, is substantially free of antibodies that specifically bind antigens other than the specific antigen). An isolated antibody can be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody” is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies can be produced by a hybridoma that includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VI. regions of the recombinant antibodies are sequences that, while derived from and related to human germline VH and VI. sequences, may not naturally exist within the human antibody germline repertoire in vivo.

The term “isotype” refers to the antibody class (e.g., IgM or IgG1) that is encoded by the heavy chain constant region genes. The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody. The term “humanized antibody” is intended to refer to antibodies in which CDR sequences derived from the germline of another mammalian species, such as a mouse, have been grafted onto human framework sequences. Additional framework region modifications can be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody. The term can also refer to an antibody in which its variable region sequence or CDR(s) is derived from one source (e.g., an IgA1 antibody) and the constant region sequence or Fc is derived from a different source (e.g., a different antibody, such as an IgG, IgA2, IgD, IgE or IgM antibody).

“Single chain antibodies” or “scFvs” are Fv molecules in which the heavy and light chain variable regions have been connected by a flexible linker to form a single polypeptide chain, which forms an antigen-binding region. scFvs are discussed in detail in WO 88/01649 and U.S. Pat. Nos. 4,946,778 and 5,260,203, the disclosures of which are incorporated by reference.

A “domain antibody” or “single chain immunoglobulin” is an immunologically functional immunoglobulin fragment containing only the variable region of a heavy chain or the variable region of a light chain. Examples of domain antibodies include Nanobodies™In some instances, two or more VH regions are covalently joined with a peptide linker to create a bivalent domain antibody. The two VH regions of a bivalent domain antibody may target the same or different antigens.

As used herein, the term “affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including those described herein.

As used herein, a protein that “specifically binds to GIPR” refers to a protein that binds to a human GIPR when the dissociation constant (KD) is ≤10⁻⁶ M as measured via a surface plasma resonance technique (e.g., BIACore, GE-Healthcare Uppsala, Sweden) or Kinetic Exclusion Assay (KinExA, Sapidyne, Boise, Id.). Preferably, the protein (e.g., antibody) binds to the GIPR with “high affinity”, namely with a KD of 1×10⁻⁷ M or less, more preferably 5×10⁻⁸ M or less, more preferably 3×10⁻⁸ M or less, more preferably 1×10⁻⁸ M or less, more preferably 5×10⁻⁹ M or less or even more preferably 1×10⁻⁹ M or less. The term “does not substantially bind” to a protein or cells, as used herein, means does not bind or does not bind with a high affinity to the protein or cells, i.e., binds to the protein or cells with a KD of 1×10⁻⁶ M or more, more preferably 1×10⁻⁵ M or more, more preferably 1×10⁻⁴ M or more, more preferably 1 x 10-3 M or more, even more preferably 1×10⁻² M or more.

The term “Kassoc” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “KD,” as used herein, is intended to refer to the dissociation constant, which is obtained from the ratio of Kd to Ka (i.e., Kd/Ka) and is expressed as a molar concentration (M). KD values for antibodies can be determined using methods well established in the art. A preferred method for determining the KD of an antibody is by using surface plasmon resonance, preferably using a biosensor system such as a Biacore® system.

The term “epitope” as used herein refers to an antigenic determinant that interacts with a specific antigen-binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immune-precipitation assays, wherein overlapping or contiguous peptides from a GIPR protein are tested for reactivity with a given antibody. Methods of determining spatial conformation of epitopes include techniques in the art and those described herein, for example, x-ray crystallography and 2-dimensional nuclear magnetic resonance (see, e.g. , Epitope Mapping Protocols in Methods in Molecular Biology, Vol. 66, G. E. Morris, Ed. (1996)).

The term “epitope mapping” refers to the process of identification of the molecular determinants for antibody-antigen recognition.

The term “binds to an epitope” or “recognizes an epitope” with reference to an antibody or antibody fragment refers to continuous or discontinuous segments of amino acids within an antigen. Those of skill in the art understand that the terms do not necessarily mean that the antibody or antibody fragment is in direct contact with every amino acid within an epitope sequence.

The term “binds to the same epitope” with reference to two or more antibodies means that the antibodies bind to the same, overlapping or encompassing continuous or discontinuous segments of amino acids. Those of skill in the art understand that the phrase “binds to the same epitope” does not necessarily mean that the antibodies bind to or contact exactly the same amino acids. The precise amino acids that the antibodies contact can differ. For example, a first antibody can bind to a segment of amino acids that is completely encompassed by the segment of amino acids bound by a second antibody. In another example, a first antibody binds one or more segments of amino acids that significantly overlap the one or more segments bound by the second antibody. For the purposes herein, such antibodies are considered to “bind to the same epitope.”

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90% or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance). Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

As used herein, the term “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+or CD8+T cell, or the inhibition of a Treg cell.

The term “detectable label” as used herein refers to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof that is capable of exhibiting fluorescence in the detectable range.

As used herein, the term “subject” refers to an animal. Preferably, the animal is a mammal, such a human or a non-human amamal. A subject also refers to for example, primates (e.g., humans), cows, sheep, goats, horses, dogs, cats, rabbits, rats, mice, fish, birds and the like. In a preferred embodiment, the subject is a human.

As used herein, the term “treating” or “treatment” of any disease or disorder refers in one embodiment, to ameliorating the disease or disorder (i.e., arresting or reducing the development of the disease or at least one of the clinical symptoms thereof). In another embodiment, “treating” or “treatment” refers to ameliorating at least one physical parameter, which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with the disease state (e.g., fatty liver, diabetes, obesity, dyslipidemia, elevated glucose levels, elevated insulin levels or diabetic nephropathy). In some embodiments, the effective amount is a therapeutically effective amount or a prophylactically effective amount. When applied to an individual active ingredient, administered alone, the term refers to that ingredient alone. When applied to a combination, the term refers to combined amounts of the active ingredients that result in the therapeutic effect, whether administered in combination, serially or simultaneously.

A “therapeutically effective amount” is an amount sufficient to remedy a disease state or symptoms, particularly a state or symptoms associated with the disease state, or otherwise prevent, hinder, retard or reverse the progression of the disease state or any other undesirable symptom associated with the disease in any way whatsoever.

A “prophylactically effective amount” is an amount of a pharmaceutical composition that, when administered to a subject, will have the intended prophylactic effect, e.g., preventing or delaying the onset (or reoccurrence) of the disease state, or reducing the likelihood of the onset (or reoccurrence) of the disease state or associated symptoms. The full therapeutic or prophylactic effect does not necessarily occur by administration of one dose, and may occur only after administration of a series of doses. Thus, a therapeutically or prophylactically effective amount may be administered in one or more administrations.

As used herein, the term “pharmaceutically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a pharmaceutically acceptable carrier or excipient is preferably suitable for topical formulation. The term includes, but is not limited to, a solvent, a stabilizer, a solubilizer, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bioadhesive polymer, or a combination thereof. The use of such agents for the formulation of pharmaceutically active substances is well known in the art (see, for example, “Remington's Pharmaceutical Sciences”, E. W. Martin, 18th Ed., 1990, Mack Publishing Co.: Easton, PA, which is incorporated herein by reference in its entirety).

As used herein, the term “a,” “an,” “the” and similar terms used in the context of the present invention (especially in the context of the claims) are to be construed to cover both the singular and plural unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to within 10%, preferably within 5%, and more preferably within 1% of a given value or range. Alternatively, the term “about” refers to within an acceptable standard error of the mean, when considered by one of ordinary skill in the art.

### EXAMPLES

### Example 1 Generation of Anti-GIPR Monoclonal Antibodies

Antigen generation: The extracellular domain of human GIPR (1-129, SEQ ID NO: 87) Fc fusion protein (Fc-huGIPR ECD) was prepared. DNA encoding this fusion protein was synthesized and inserted into a mammalian cell expression vector. The plasmid expressing Fc-huGIPR ECD was transiently transfected in CHO cells under serum-free suspension culture using CHO cell transfection kit (Zhuhai Kairui Biotech Cat #K70201). Cells were harvested seven days post transfection, and Fc-huGIPR ECD was then purified by Protein A affinity chromatography.

CHO cells transiently expressing huGIPR: CHO cells were transfected with a plasmid expressing full-length huGIPR in suspension culture using CHO cell transfection kit (Zhuhai Kairui Biotech Cat #K70201). By 24 hours post transfection, cells were harvested and resuspended in cell freezing media. The cells were aliquoted into cell freezing vials and kept frozen in liquid nitrogen freezer. Frozen vials were retrieved and thawed immediately in 37° C. water bath. The cells were diluted in a cell culture medium supplemented with 5% fetal bovine serum, seeded in 96 well plates at a density of 1×10⁵ cells/well and incubated at 37° C. with 5% CO2 for 24 hours before used for binding assay to detect human GIPR specific antibodies. CHO cells with mock transfection were used as negative control.

Immunization: Balb/c mice were immunized with Fc-huGIPR ECD protein mixed with Freund's adjuvants. Mice were immunized either weekly or monthly. After at lest two boost doses post initial immunization, sera were collected and specific titers were determined by the binding assay using CHO cells transiently expressing human GIPR.

Hybridoma fusion and culture: Animals exhibiting satisfactory titers were identified, and lymphocytes were obtained from draining lymph nodes and spleens. Lymphocytes were dissociated from lymphoid tissues in a suitable medium to release the cells from the tissues, and suspended in a suitable medium.

B-cells were fused with myeloma cell line SP2/O-AG1 cells (ATCC CRL 1580) at a ratio of 10:1. Fusion was performed with using PEG. The fused cells were gently pelleted (300×g 10 minutes) and resuspended in selection media containing hypoxanthine-methotrexate-thymidine [HMT]. Cells were distributed into 96-well plates using standard techniques to maximize clonality of the resulting colonies. After several days of culture, the hybridoma supernatants were collected and subjected to screening assays, which was a binding assay using CHO cells transiently expressing human GIPR. Positive cells were further selected and subjected to standard cloning and subcloning techniques. Clonal lines were expanded in vitro, and the secreted mouse antibodies obtained for analysis.

Selection of GIPR specific binding antibodies by whole cell binding assay: Hybridoma supernatants were screened for GIPR-specific antibodies by whole cell binding assay. Briefly, CHO cells were transfected with a plasmid expressing full-length huGIPR. 24 hours post transfection, the cells were plated in 96-well plates and incubated for 24 hours. Hybridoma conditioned media were added to each plate and incubated for 1 hour at 37° C. After three washes with cell culture medium, the cells were then fixed with ethanol, and plates were dried overnight. A blocking buffer (PBS with 0.1% TWEEN 20 and 1% BSA) were added 200 ul/well and incubated for 1 hour at 37° C. After the blocking buffer was removed, detection antibody (goat anti-mouse IgG HRP conjugate) was added and incubated for 1 hour at 37° C. 3,3′,5,5′-Tetramethylbenzidine (TMB) was then added for color detection. CHO cells with mock transfection were used as counterscreen. Any clones with specific positive signal with GIPR expressing CHO cells were selected for subcloning. Total 87 clones were tested positive in the binding assay. Further subcloning and retesting identified 7 positive clones. They were then sequenced.

### Example 2 In Vitro Activity of Selected Anti-GIPR Antibodies

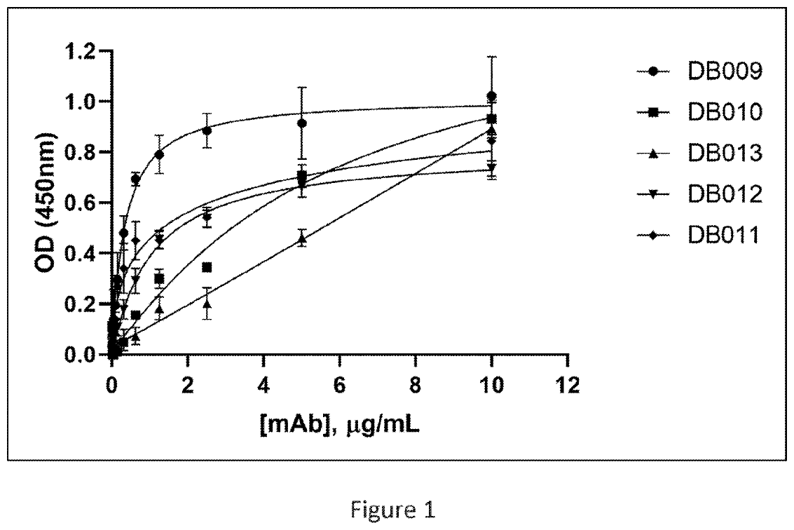

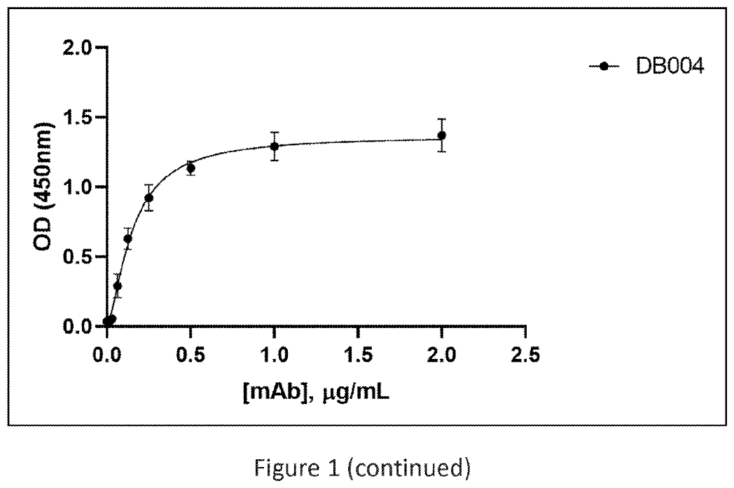

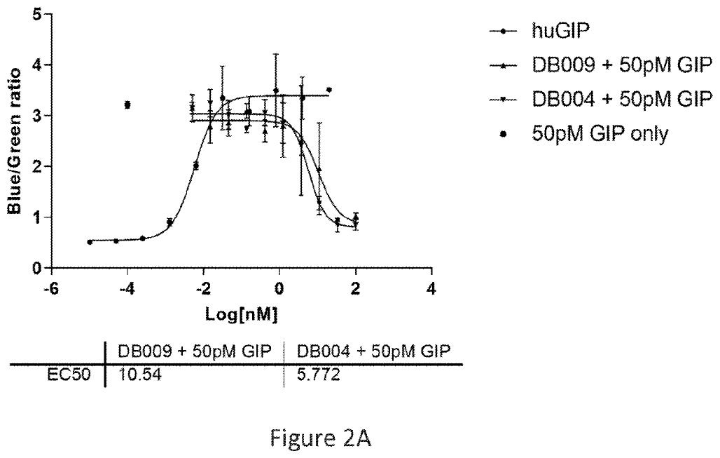

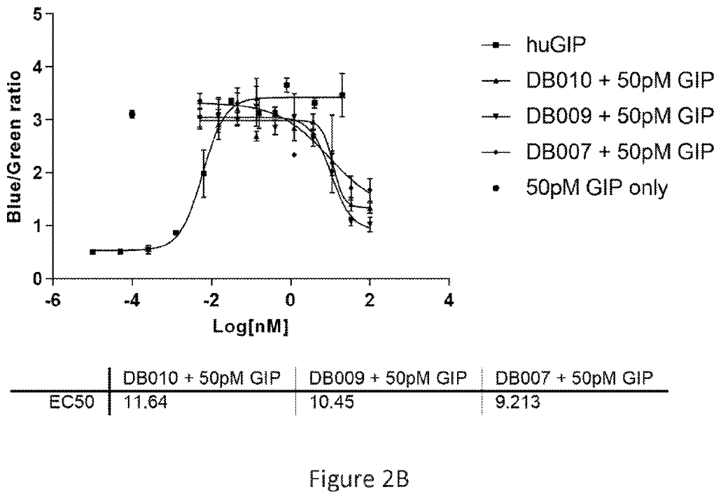

The anti-GIPR monoclonal antibodies obtained were tested in the GeneBLAzer® GIPR-CRE-bla HEK 293T cell-based assay (INVITROGEN, THERMO FISHER SCIENTIFIC). GeneBLAzer® GIPR-CRE-bla HEK 293T cells contain the human GIPR stably integrated into the CellSensor® CRE-bla HEK 293T cell line, while CellSensor® CRE-bla HEK 293T contains a beta-lactamase reporter gene under control of a CRE response element. This assay uses mammalian-optimized bla reporter gene combined with a FRET-enabled substrate to provide reliable and sensitive detection in cells expressing human GIPR. This assay was performed according to manufacturer' protocol. The results are shown in FIG. 1.

Briefly, the day before the assay, cells were detached with 5 mM EDTA and seeded into 96-well plate with 50,000 cells/100 μL per well. For the agonist mode, 25 μL of ligand (human GIP, 5× of final concentration) was added per well. In the antagonist mode, 12.5 μL of antibody (or medium, at 10× final concentration) was added into each well, and the cells were cultured at 37° C. for 30 minutes. 12.5 μL of ligand (human GIP, with appropriate concentration prepared with assay buffer) were added to each well. The cells were then incubated at 37° C. for 5 hours. 25 μL per well of 6× substrate mix containing CCF4-AM was then added to cells and incubated for 2 hours at room temperature in the dark. The signal of plate was read in a fluorescent plate reader. Scan 1 was to measure fluorescence in the Blue channel with excitation filter: 409/20 nm, and emission filter: 460/40 nm, and scan 2 was to measure fluorescence in the Green channel with excitation filter: 409/20 nm, and emission filter: 530/30 nm. Data was analyzed for background subtraction and ratio calculation according to manufacturer's recommendation.

TABLE 1
Beta-lactamase reporter assay activity
summary for- selected GIPR antibodies.

| GIPR mAb ID | IC50 (nM) |
|---|---|
| DB004 | 3.3 |
| DB006 | 4.1 |
| DB007 | 9.2 |
| DB009 | 6.3 |
| DB010 | 11.6 |

### Example 3 In Vivo Activity of Selected Anti-GIPR Antibodies

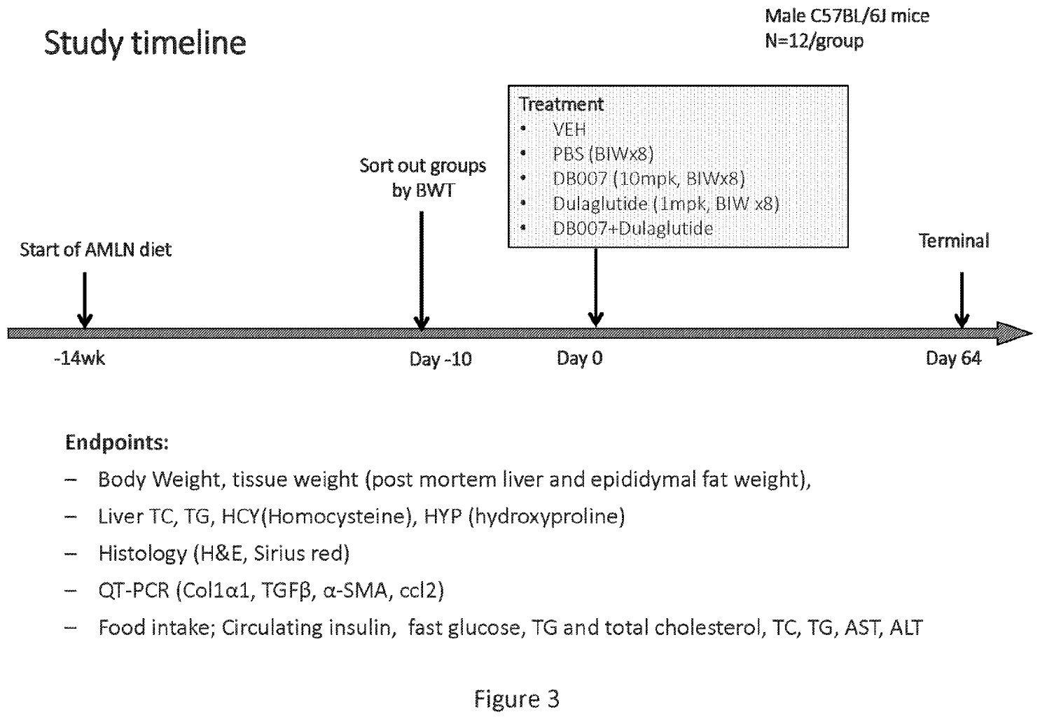

The study scheme is illustrated in FIG. 3. Five-week old male C57BL/6J mice were purchased. After arrival, the mice were single housed. Some were fed on ALMN diet (Research Diet D09100301, 40% fat with mostly PRIMEX) for 14 weeks, while some were fed on chow diet as age-matched controls. The animals following 14-week high fat treatment were further divided into four subgroups based on their similar bodyweight and blood glucose levels (Group B, C, D and E, 10/group). Detailed study design is described as following. Group A was injected with vehicle control under chow diet, Group B was injected with vehicle control under AMLN diet, Group C was injected with Anti-GIPR mAb DB007 under AMLN diet, Group D was injected with Dulaglutide under AMLN diet, and Group E was injected with DB007+dulaglutide combo under AMLN diet. DB007 (10 mg/kg), dulaglutide (1 mg/kg) and vehicle were dosed twice weekly via intraperitoneal injections for 9 weeks. Animals were monitored for any changes in the general conditions including abnormal behaviors by routine observations once daily and the body weight was measured twice weekly over the course of the study.

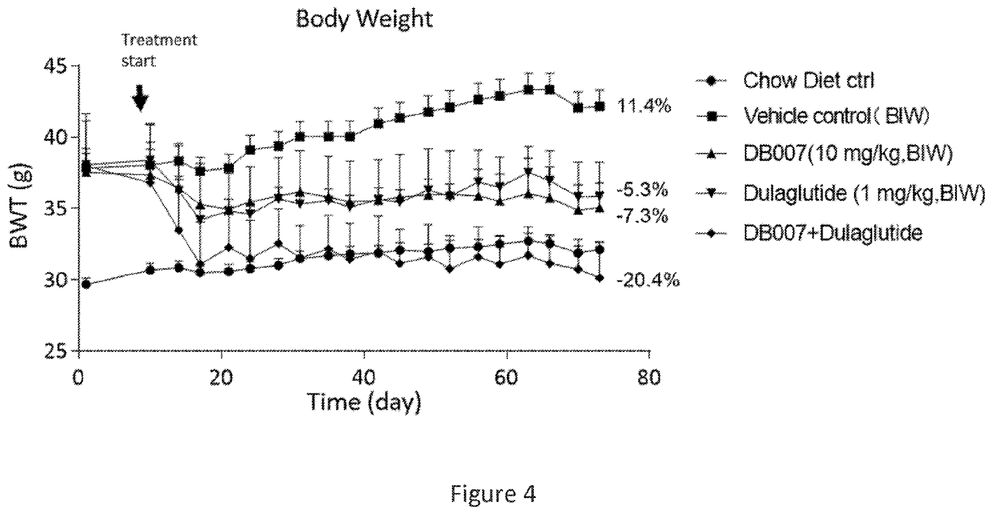

Body Weight. Body weight for each group of animals was determined at baseline, and twice per week during the study. As shown in FIG. 4, the body weight of vehicle group under ALMN diet had significantly higher body weight than chow diet control group. Anti-GIPR mAb DB007 and dulaglutide reduced body weight compared to the vehicle control group. In addition, the DB007 and dulaglutide combo group reduced body weight more dramatically and the body weight is close to chow diet control group. All the body weight difference between each group receiving DB007, dulaglutide or combo and the vehicle control group were statistically significant, and the body weight changes were maintained throughout the treatment period.

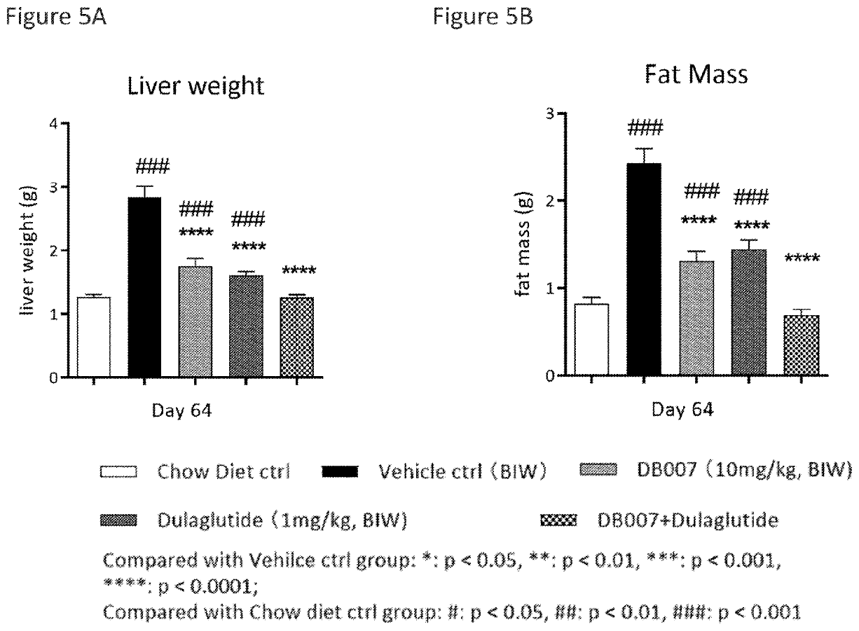

Liver and Fat Weight. At termination of the study, the liver and epididymal fat were excised and weighed. As shown in FIG. 5, the liver and epididymal fat weight of vehicle group under ALMN diet were significantly higher than the liver and epididymal fat weight of the chow diet control group. Anti-GIPR mAb DB007 and dulaglutide reduced liver and fat weight compared to the vehicle control group. In addition, the DB007 and dulaglutide combo group reduced more liver and fat weight than themselves alone.

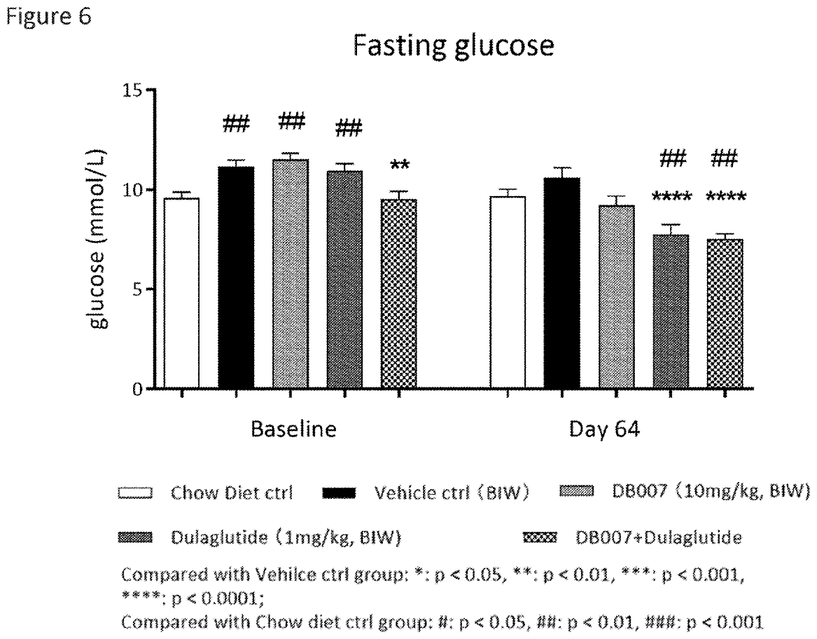

Glucose. Glucose levels were obtained at baseline, and at Day 64 (4 h fast) of the study. As shown in FIG. 6, the fasting glucose level of the vehicle group under ALMN diet is mildly higher than the chow diet control group. Anti-GIPR mAb DB007 did not significantly reduced fasting glucose level. However, dulaglutide and combo treatment did significantly reduce fasting glucose level than vehicle group.

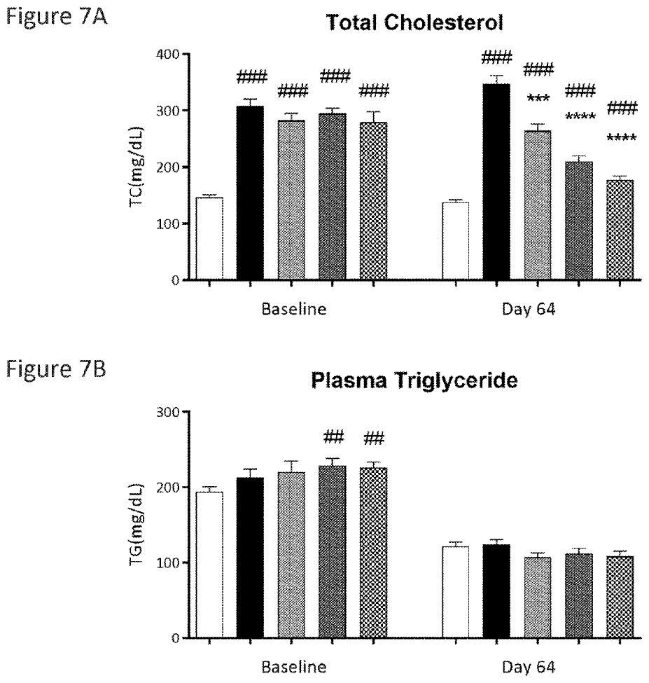

Lipids. Blood was collected at Day 64 at termination of the study for determination of total cholesterol, triglycerides. As shown in FIG. 7, the total cholesterol of vehicle group under ALMN diet are significantly higher than chow diet control group. At terminal bleed, the total cholesterol levels were significantly lower in the treatment groups with the combo treatment trending to the lowest level. However, no significant changes in triglyceride were observed.

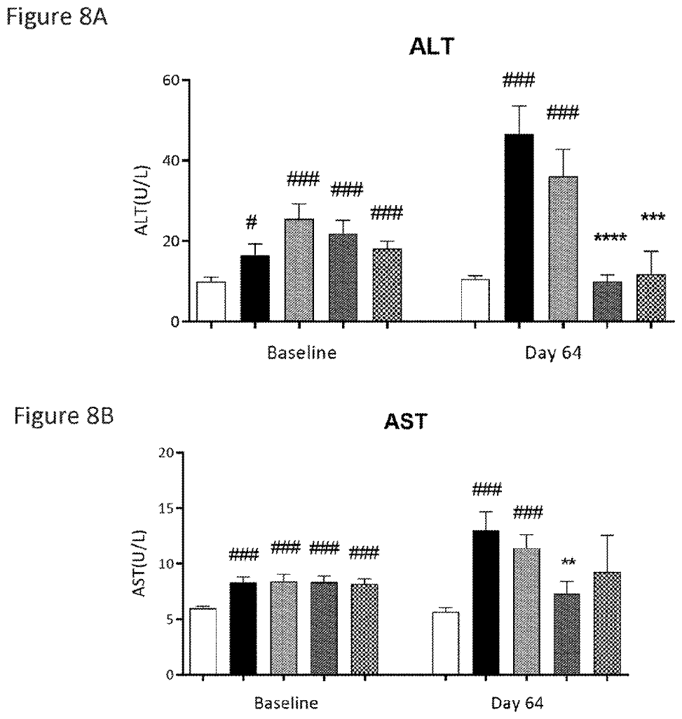

Liver enzymes. Blood was collected at Day 64 at termination of the study for determination of liver enzyme ALT and AST. As shown in FIG. 8, the ALT level of vehicle group under ALMN diet was significantly higher than chow diet control group at terminal bleed. The ALT levels were significantly lower in the dulaglutide and DB007 and dulaglutide combo treatment groups. The AST level of vehicle group under ALMN diet was also significantly higher than chow diet control group at terminal bleed. However, only dulaglutide alone significantly reduced AST level.

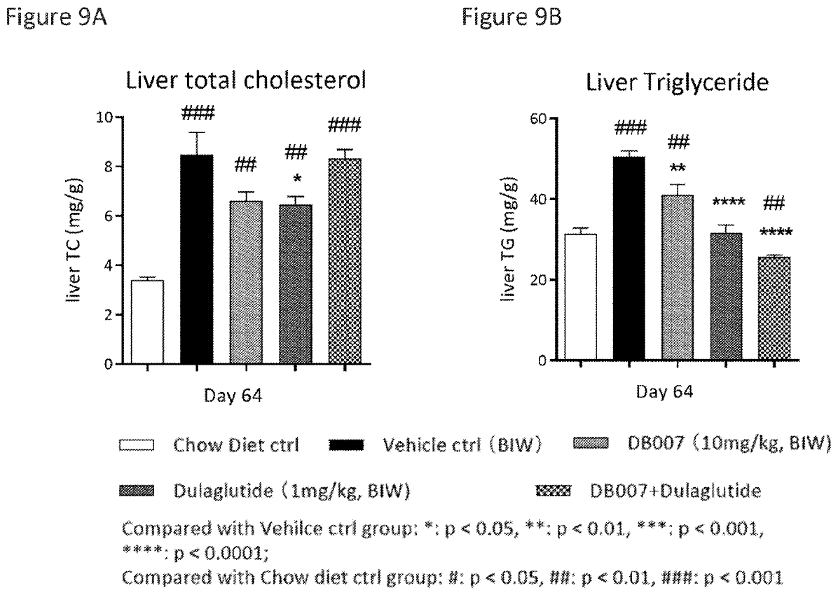

Liver lipid profile. Liver total cholesterol and triglyceride were measured at termination of the study. As shown in FIG. 9, the liver total cholesterol of vehicle group under ALMN diet was significantly higher than that of the chow diet control group. DB007 and dulaglutide only mildly reduced liver total cholesterol level. The liver triglyceride of vehicle group under ALMN diet was also significantly higher than that of the chow diet control group. Both DB007 and dulaglutide reduced liver triglyceride levels significantly, and combo treatment reduced liver triglyceride even further.

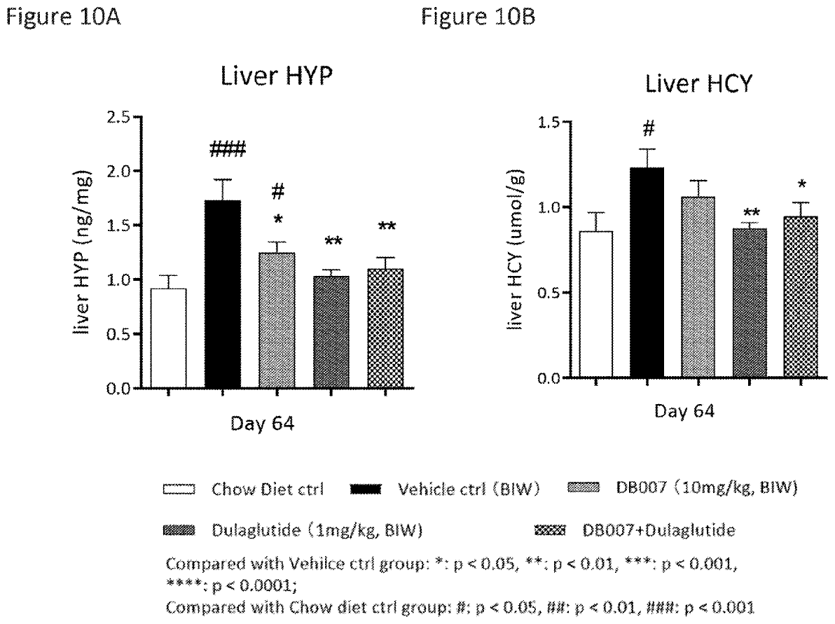

Liver biomarkers. Homocysteine (HCY), formed as an intermediary in methionine metabolism, is a sulfur-containing amino acid. Liver plays a central role in the metabolism of methionine and HCY. Increased HCY metabolism may occur in liver damage. Hydroxyproline (HYP) is one of the most abundant amino acids present in collagen following hydroxylation of proline moiety. Its level in liver tissue could signify correctly the rate and progression of liver fibrogenesis. HYP could be a biomarker in chronic liver diseases with severe fibrosis. Therefore, Liver HCY and HYP levels were measured at termination of the study. As shown in FIG. 10, the liver HCY and HYP levels of the vehicle group under ALMN diet were significantly higher than those of the chow diet control group. DB007, dulaglutide and combo treatments all significantly reduced liver HCY and HYP levels to a normal level.

Expression levels of a number of genes in liver were also measured using quantitative PCR. Alpha-smooth muscle actin (α-SMA) expression is a reliable marker of hepatic stellate cells activation which precedes fibrous tissue deposition. It can be useful to identify early stages of hepatic fibrosis and monitoring the efficacy of the therapy. Transforming growth factor-β (TGF-β) is a central regulator in chronic liver disease contributing to all stages of disease progression. Its expression is also useful to monitor the efficacy of the therapy. Collagen type 1 alpha 1 (Col1a1) gene encodes a protein that is the major component of type I collagen, a major component of fibrosis. CCL2 is an important inflammatory chemokine involved in monocyte recruitment to inflamed tissues. Those genes were measured at termination of the study.

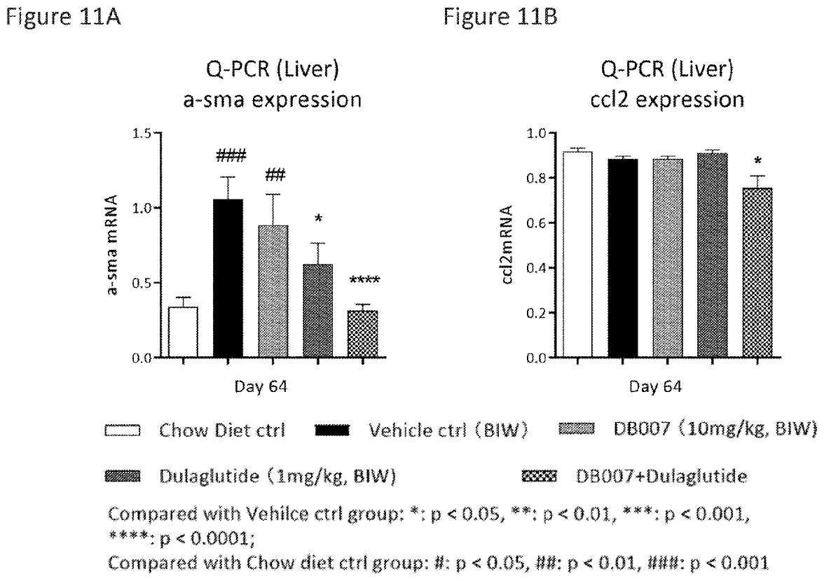

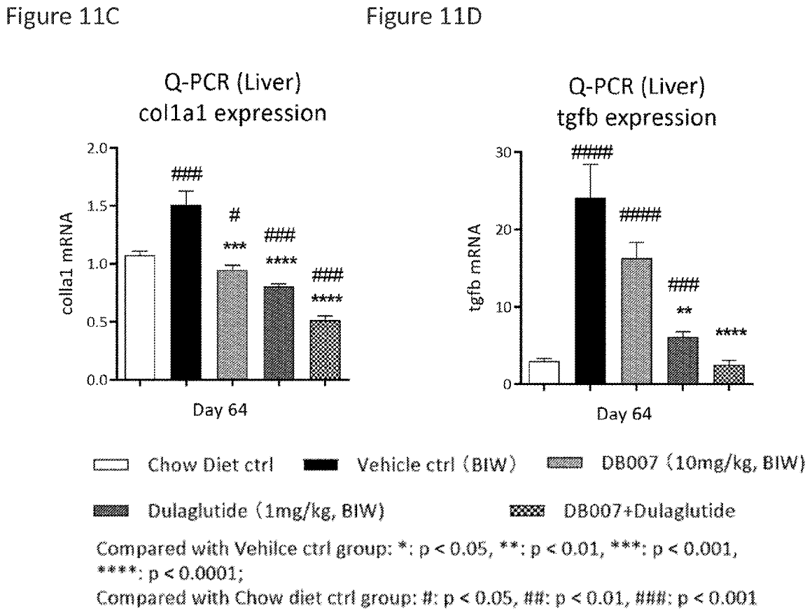

As shown in FIG. 11, the liver α-SMA, col1a1, and TGFB gene expressions of vehicle group under ALMN diet were significantly higher than those of the chow diet control group, whereas ccl2 expression did not change. DB007 and dulaglutide alone reduced the expression of α-SMA, col1a1, and TGFβ, and the combo therapy further reduced their expressions. Interestingly, the combo therapy not only completely normalized the expression of α-SMA and TGFβ to that of chow diet control, but also reduced col1a1 and ccl2 expressions lower than that of chow diet control.

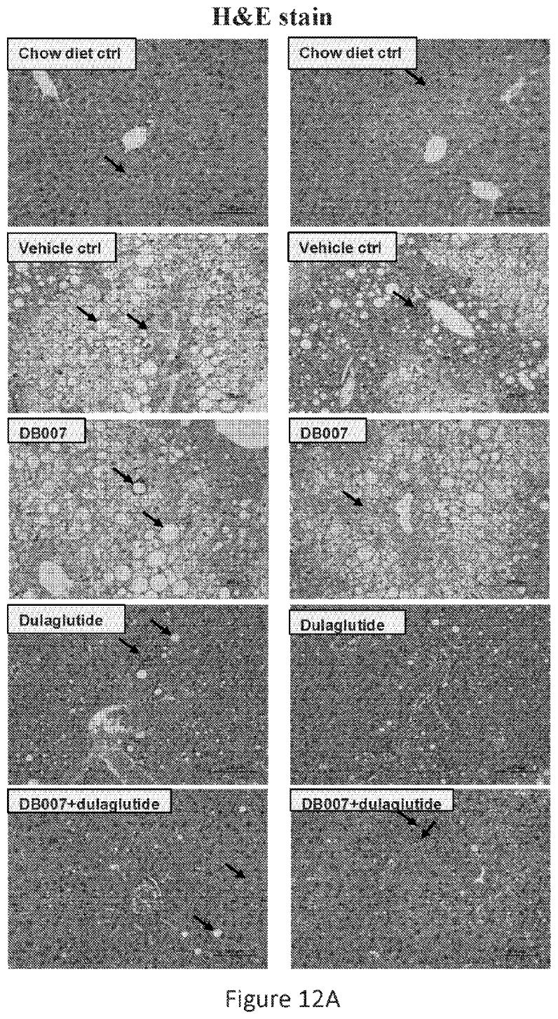

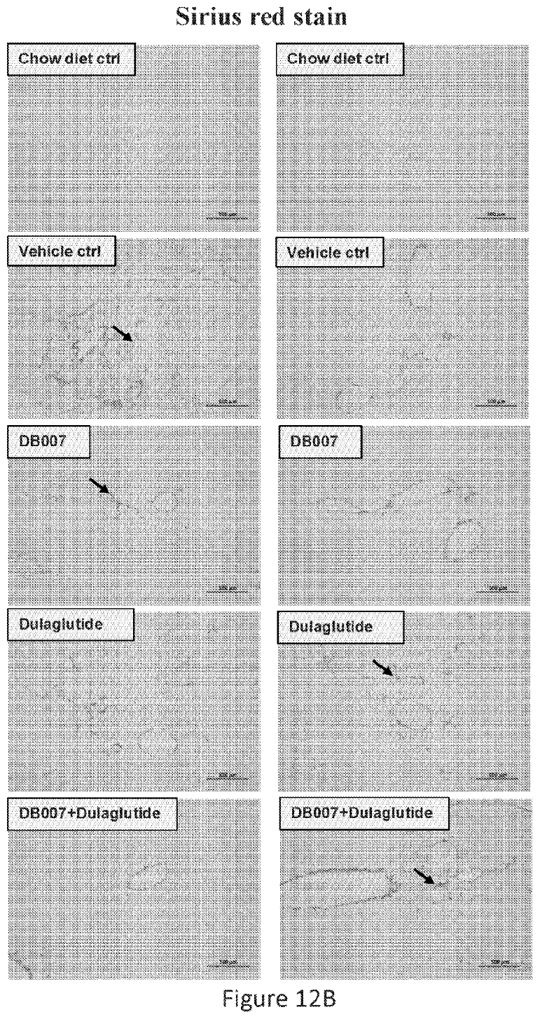

Liver histological analysis. At termination of the study, part of liver tissues was fixed in 10% formaldehyde and embedded in paraffin. Paraffin-embedded sections were stained with hematoxylin and eosin (H&E) or Sirius red. The NAFLD activity and fibrosis stage were scored. As shown in FIG. 12, AMLN diet significantly increased liver steatosis as demonstrated by increased lipid droplets in H&E staining, and slightly increased fibrosis as shown in Sirius red staining; DB007 slightly reduced liver steatosis and fibrosis, and dulaglutide alone significantly reduced liver steatosis, and did have much impact on fibrosis. The combo treatment completely normalized liver steatosis to the level of chow diet control, and reduced liver fibrosis. The NAFLD and Fibrosis scores are shown below.

TABLE 2
NAFLD score and Fibrosis score.

| | NAFLD | | Fibrosis | |
|---|---|---|---|---|
| Group | score | NASH | score | Fibrosis |
| Chow diet ctrl | 0-3 | Non-NASH | 0 | no obvious collagen fibrosis |
| Vehicle | 3-5 | Probable or definite | 0-2 | local collagen fibrosis |
| ctrl (BIW × 9 | | NASH | | observed (black arrow) |
| DB007 (10 | 1-5 | Probable or definite | 0-2 | Some collagen fibrosis |
| mg/kg, BIW × 9) | | NASH | | observed near central vein |
| | | | | (black arrow) |
| Dulaglutide (1 | 1-3 | Non-NASH | 0-2 | Some collagen fibrosis |
| mg/kg, BIW × 9) | | | | observed near central vein |
| | | | | (black arrow) |
| DB007 + | 0-3 | Non-NASH | 0-2 | Some collagen fibrosis |
| dulaglutide | | | | observed near central vein |
| | | | | (black arrow) |

Data Analysis. Data were analyzed using GRAPHPAD PRISM program (GRAPHPAD, La Jolla, CA). Statistical analysis was performed using either one-way or two-way ANOVA. Treatment group compared with vehicle control group: *: p<0.05, **: p<0.01, ***: p<0.001. Vehicle and treatment groups compared with chow diet control group: #: p<0.05, ##: p<0.01, ###: p<0.001. P<0.05 was considered significant.

### REFERENCES

1. J. E. Campbell, D. J. Drucker, Pharmacology, physiology, and mechanisms of incretin hormone action. Cell Metab 17, 819-837 (2013).
2. B. Ahren, The future of incretin-based therapy: novel avenues—novel targets. Diabetes Obes Metab 13 Suppl 1, 158-166 (2011).
3. M. A. Nauck et al., Preserved incretin activity of glucagon-like peptide 1 [7-36 amide] but not of synthetic human gastric inhibitory polypeptide in patients with type-2 diabetes mellitus. J Clin Invest 91, 301-307 (1993).
4. J. M. Falko, S. E. Crockett, S. Cataland, E. L. Mazzaferri, Gastric inhibitory polypeptide (GIP) stimulated by fat ingestion in man. J Clin Endocrinol Metab 41, 260-265 (1975).
5. A. B. Damholt, H. Kofod, A. M. Buchan, Immunocytochemical evidence for a paracrine interaction between GIP and GLP-1-producing cells in canine small intestine. Cell Tissue Res 298, 287-293 (1999).
6. T. J. Kieffer, C. H. McIntosh, R. A. Pederson, Degradation of glucose-dependent insulinotropic polypeptide and truncated glucagon-like peptide 1 in vitro and in vivo by dipeptidyl peptidase IV. Endocrinology 136, 3585-3596 (1995).
7. A. Couvineau, M. Laburthe, The family B1 GPCR: structural aspects and interaction with accessory proteins. Curr Drug Targets 13, 103-115 (2012).
8. C. C. Tseng, X. Y. Zhang, Role of G protein-coupled receptor kinases in glucose-dependent insulinotropic polypeptide receptor signaling. Endocrinology 141, 947-952 (2000).
9. T. B. Usdin, E. Mezey, D. C. Button, M. J. Brownstein, T. I. Bonner, Gastric inhibitory polypeptide receptor, a member of the secretin-vasoactive intestinal peptide receptor family, is widely distributed in peripheral organs and the brain. Endocrinology 133, 2861-2870 (1993).
10. A. Lund, T. Vilsboll, J. I. Bagger, J. J. Holst, F. K. Knop, The separate and combined impact of the intestinal hormones, GIP, GLP-1, and GLP-2, on glucagon secretion in type 2 diabetes. Am J Physiol Endocrinol Metab 300, E1038-1046 (2011).
11. S. J. Kim, C. Nian, C. H. McIntosh, Activation of lipoprotein lipase by glucose-dependent insulinotropic polypeptide in adipocytes. A role for a protein kinase B, LKB1, and AMP-activated protein kinase cascade. J Biol Chem 282, 8557-8567 (2007).
12. E. Faivre, A. Hamilton, C. Holscher, Effects of acute and chronic administration of GIP 12. analogues on cognition, synaptic plasticity and neurogenesis in mice. Eur J Pharmacol 674, 294-306 (2012).
13. S. Gremlich et al., Cloning, functional expression, and chromosomal localization of the human pancreatic islet glucose-dependent insulinotropic polypeptide receptor. Diabetes 44, 1202-1208 (1995).
14. K. Miyawaki et al., Inhibition of gastric inhibitory polypeptide signaling prevents obesity. Nat Med 8, 738-742 (2002).
15. S. I. Berndt et al., Genome-wide meta-analysis identifies 11 new loci for anthropometric traits and provides insights into genetic architecture. Nat Genet 45, 501-512 (2013).
16. Y. Okada et al., Common variants at CDKAL1 and KLF9 are associated with body mass index in east Asian populations. Nat Genet 44, 302-306 (2012).
17. R. Saxena et al., Genetic variation in GIPR influences the glucose and insulin responses to an oral glucose challenge. Nat Genet 42, 142-148 (2010).
18. E. K. Speliotes et al., Association analyses of 249,796 individuals reveal 18 new loci associated with body mass index. Nat Genet 42, 937-948 (2010).
19. W. Wen et al., Meta-analysis identifies common variants associated with body mass index in east Asians. Nat Genet 44, 307-311 (2012).
20. D. J. Drucker, Enhancing incretin action for the treatment of type 2 diabetes. Diabetes Care 26, 2929-2940 (2003).
21. M. A. Nauck, J. J. Holst, B. Willms, W. Schmiegel, Glucagon-like peptide 1 (GLP-1) as a new therapeutic approach for type 2-diabetes. Exp Clin Endocrinol Diabetes 105, 187-195 (1997).
22. S. Dhillon, Semaglutide: First Global Approval. Drugs 78, 275-284 (2018).
23. T. Vilsboll, M. Christensen, A. E. Junker, F. K. Knop, L. L. Gluud, Effects of glucagon-like peptide-1 receptor agonists on weight loss: systematic review and meta-analyses of randomised controlled trials. BMJ 344, d7771 (2012).

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein in their entireties. are incorporated by reference

1. A method of treating a subject with a fatty liver disease, the method comprising administering to the subject a therapeutically effective amount of an antigen binding protein that specifically binds to a protein having an amino acid sequence having at least 90% amino acid sequence identity to an amino acid sequence of a gastric inhibitory peptide receptor (GIPR).

2-3. (canceled)

4. An isolated antibody or antigen-binding fragment thereof that specifically binds to human gastric inhibitory peptide receptor (GIPR), comprising:
(i) a light chain or a light chain variable region that comprises LCDR1, LCDR2 and LCDR3 comprising the respective sequences of a LCDR set selected from the group consisting of SEQ ID NOs: 1-3, SEQ ID NOs: 7-9, SEQ ID NOs: 7, 2, and 3, SEQ ID NOs: 10, 2, and 3, SEQ ID NOs: 12-14, SEQ ID NOs: 18-20, SEQ ID NOs: 24-26, SEQ ID NOs: 30-32, and SEQ ID NOs: 36-38, and/or
(ii) a heavy chain or a heavy chain variable region that comprises HCDR1, HCDR2, and HCDR3 comprising the respective sequences of a HCDR set selected from the group consisting of SEQ ID NOs: 4-6, SEQ ID NOs: 4, 11, and 6, SEQ ID NOs: 15-17, SEQ ID NOs: 21-23, SEQ ID NOs: 27-29, SEQ ID NOs: 33-35, SEQ ID NOs: 39-41, SEQ ID NOs: 39, 40, and 42, SEQ ID NOS:
39, 40, and 43, and SEQ ID NOs: 39, 40, and 44.

5. The isolated antibody or the antigen-binding fragment thereof of claim 4, wherein
the light chain variable region comprises a sequence selected from the group consisting of SEQ ID NOs: 45, 47-50, 57, 59, 61, 63, 65, and 67-69, and
the heavy chain variable region comprises a sequence selected from the group consisting of SEQ ID NOs: 46, 51-56, 58, 60, 62, 64, 66, and 70-73.

6-8. (canceled)

9. The isolated antibody or the antigen-binding fragment thereof of claim 4, wherein the light chain comprises a sequence selected from the group consisting of SEQ ID NOs: 74 and 78, and the heavy chain comprises a sequence selected from the group consisting of SEQ ID NOs: 76 and 80.

10-12. (canceled)

13. The isolated antibody or the antigen-binding fragment thereof of claim 4, wherein the antibody or fragment is conjugated to one or more of a therapeutic agent, a polymer, a detectable label, or an enzyme.

14. The isolated antibody or the antigen-binding fragment thereof of claim 13, wherein the antibody or fragment is conjugated to a GLP-1 sequence.

15. The isolated antibody or the antigen-binding fragment thereof of claim 14, wherein the GLP-1 sequence comprises the sequence of SEQ ID NO: 82 or a functional variant thereof.

16-19. (canceled)

20. The isolated antibody or the antigen-binding fragment thereof of claim 19, wherein the heavy chain comprises the sequence of SEQ ID NO: 85 or 87, or the light chain comprises the sequence of SEQ ID NO: 86 or 89, or wherein the light chain and the heavy chain comprise the respective sequences of a set selected from the group consisting of SEQ ID NOs: 78 and 87, and SEQ ID NOs: 89 and 80.

21-22. (canceled)

23. An isolated nucleic acid encoding a CDR, a heavy light chain variable region, or a light chain variable region of the antibody, or antigen-binding portion thereof, of claim 4, or an expression vector comprising the isolated nucleic acid.

24. (canceled)

25. A host cell comprising the nucleic acid of claim 23 or an expression vector comprising the nucleic acid.

26. A method of preparing an antibody, or antigen-binding portion thereof, comprising:
obtaining a cultured host cell comprising a vector comprising a nucleic acid sequence encoding a CDR, a heavy chain variable region, or a light chain variable region of the antibody or antigen binding portion thereof of claim 4;
culturing the cell in a medium under conditions permitting expression of a polypeptide encoded by the vector and assembling of an antibody or fragment thereof, and
purifying the antibody or fragment from the cultured cell or the medium of the cell.

27. A pharmaceutical composition comprising the antibody or the antigen-binding fragment thereof of claim 4 and a pharmaceutically acceptable carrier.

28. A method of treating a subject with a metabolic disorder, the method comprising administering to a subject in need thereof a therapeutically effective amount of the antibody or the antigen-binding fragment thereof of claim 4, or a therapeutically effective amount of a composition comprising the antibody or the antigen-binding fragment thereof of and a pharmaceutically acceptable carrier, or,
the method comprising the steps of: (a) administering to the subject an effective amount of (i) a nucleic acid encoding a CDR, a heavy light chain variable region, or a light chain variable region of the antibody, or antigen-binding portion thereof, or (ii) an expression vector comprising the nucleic acid, or (iii) a host cell comprising the nucleic acid or the expression vector and (b) expressing the nucleic acid in the subject.

29. (canceled)

30. The method of claim 28 er 29, wherein the metabolic disorder is a disorder of fatty liver disease or a disorder of glucose metabolism.

31. The method of claim 30, wherein the fatty liver disease is a disorder of non-alcoholic fatty liver disease.

32. The method of claim 31, wherein the non-alcoholic fatty liver disease is a disorder of nonalcoholic steatohepatitis.

33. (canceled)

34. The method of claim 30, wherein the glucose metabolism disorder comprises one or more of hyperglycemia, hyperinsulinemia, glucose intolerance, insulin resistance, diabetes mellitus and obesity.

35. The method of claim 28, further comprising administering to the subject a second therapeutic agent. 36 (Original) The method of claim 35, wherein the second therapeutic agent is a Glucagon-Like Peptide-1 (GLP-1) receptor agonist.

37. The method of claim 36, wherein the GLP-1 receptor agonist is selected from the group consisting of liraglutide, exenatide, lixisenatide, dulaglutide, albiglutide, semaglutide, and taspoglutide.

38-41. (canceled)
